SEQUENCE LISTING (6 sequences)

SEQ ID NO: 1 - DNA, 1527 bp, Unknown
  Obtained from environmental sample
catatggccgaaacgaatctgcacaagaaaattaacatcgaaatgcactttgataacctggtgtccaactttgaaaaaatccgtaaactgaataacgaaaatgttgatgtcgcagacaaggtgatcccggtggttaaagcgaacgcctacaacttcggtgtttacgatatcacgaagaccttcctgtctatgaaggaaccgcagaccaaatttttcgtgtttagtctggaagaaggtattgaactgcgtaaatttttcccgtcgattgaacgcatctttgttattatgggcgcgctggaaaacgatgccgaactgtttaaaaagtataacctgacgccggtcgtgaatagcttctaccagctggaagtttgcaagaagcataacatcaagaacatcgtcctgcaattcaataccggtatgaaccgtaatggcttcgaagttaaagaactgcgcaaagtcaagcagatctgcgatagcgaaaaatttaacgtcctgatggtgatgagtcacttcgcctgttccgacaatatggcatcagaagtgaacgcttaccagatcaacgaatacaagaaggttgcacgtgtcttcaacgataaaaagattatcaagtccttcccggctacgttcggtatcctgaacttcaataccattaacggcctggtgaattcatttcgtccgggtcgcctgtattacggcttcctgccgaaggaaggttatgataaaaacctgttttcggtgtatgtttacctgagcaaagacaaggaaggcaatattatcctgccgttcggcaaagaagatggtatcgactatggcgaacaggcgtacattctggtcgataaaaataaggcctatgtgaagcaagttgactctcataaagttatcctggatacgaacgacaaaaccctggtcggtaaaaaggtgtacctgatcaagaacggcatgggttacgatgaatttaaggaacagagtaaacaagacctgctgacgtttttcggcaagatcttcaagaactgcgataagaacaagaacctgtgtaacatcaacgcgacccgtaaacgcgccaaaaaggataataagtttgtgccgaaagcaatcttcaaaaaggacgctaaaggccagtatacgcaatacaccacgaccctgtttgaaaaacgtgaagtgtccgcggatggcatcgttggttatgacgcaacccgcgctgtcaaaaagggtgataaactgggcgtgatttttaacggtcacatggacggcttctatcagccgatgtctaacaagcacgtcttcgtgtacgttaagaacaagtacaagcagctgatcccgtgtgaattctatggtctgattagctctgatcaagcggtgattaaaatcccgaacgatgaatttgacaatatcgaatacggcgccaaggttgtcgtgtttgataaaaatcacccgattacgctgttcgaacaggcaaccggtgttagtcgtgatgaactgttttatggcctggacaaaacctaccgcattgctgaacaaaaactcgag

SEQ ID NO: 2 - protein, 506 aa, Unknown
  Obtained from environmental sample
MAETNLHKKINIEMHFDNLVSNFEKIRKLNNENVDVADKVIPVVKANAYNFGVYDITKTFLSMKEPQTKFFVFSLEEGIELRKFFPSIERIFVIMGALENDAELFKKYNLTPVVNSFYQLEVCKKHNIKNIVLQFNTGMNRNGFEVKELRKVKQICDSEKFNVLMVMSHFACSDNMASEVNAYQINEYKKVARVFNDKKIIKSFPATFGILNFNTINGLVNSFRPGRLYYGFLPKEGYDKNLFSVYVYLSKDKEGNIILPFGKEDGIDYGEQAYILVDKNKAYVKQVDSHKVILDTNDKTLVGKKVYLIKNGMGYDEFKEQSKQDLLTFFGKIFKNCDKNKNLCNINATRKRAKKDNKFVPKAIFKKDAKGQYTQYTTTLFEKREVSADGIVGYDATRAVKKGDKLGVIFNGHMDGFYQPMSNKHVFVYVKNKYKQLIPCEFYGLISSDQAVIKIPNDEFDNIEYGAKVVVFDKNHPITLFEQATGVSRDELFYGLDKTYRIAEQK

SEQ ID NO: 3 - DNA, 1308 bp, Bacillus altitudinis
atgagcggttttacagcgttaagtgaagcagaattaaatgacctatatgcagcacgacaaaaagagtatgaaacgtacaaaagtaaaaacttacacttagacatgtctagagggaaaccttcaccaaaacagctcgatttatctatgggcatgctcgatgtcgtgacatcaaaggatgcaatgacagctgaggatggtacagatgtgcgaaactatggcggcttgacaggccttcctgaaacaaagaaattttttgcaagtgtgctcaatctgaagccagaacaaatcatcattggcggtaattctagcctaaatatgatgcatgacacaattgcccgtgctatgactcacggcgtatatggcagcaaaacaccttggggagagcttccaaaggtaaaattccttgcaccaagcccagggtatgatcgtcattttgccatttgtgagcattttaacatagagatgattacggtagatatgaagtcggatggacctgacatggatcaggtggaaaaattggttgcagaagatgaagccatcaaagggatttggtgtgtaccaaaatatagcaaccctgacggcattacgtattcagatgaggttgtcgaccgtcttgcttccatgcagacaaaagcagacgacttccgtattttttgggatgatgcctatgcagtccaccatctaacagatacgcctgatacgttaaaagatatttttcaagcagtagacaaagcagggcatgcaaaccgtgtgtttatgttcgcctctacttctaaaattacgttcccaggctcaggtgttgcactgatggcatctagtcaggacaacgtcagctttattcaaaaacagctatcagttcaaaccattgggccagataaaatcaatcaattaagacaccttcgtttcttcaagaatccagaaggattgaaggaacatatgaaaaagcatgcagcgattattaagccgaaatttgacctcgttctttcgatccttgatgaaaagcttggtggaacaggcatcgctgagtggcacaaaccaaatggcggatattttattagcttaaatacactcgatcattgtgcaaaagctgttgtgcaaaaagcgaaagaagccggtgtgacactaacaggtgcaggggcgacatatccttatggaaacgacccgcttgatcgtaacatccgtattgcgccaacgttcccaacgcttgaagaactagagcaggcgattgatatctttacgttatgcgttcagcttgtcagcattgaaaagctgctgtctgagaaaagtcaatcagcaccaacggtataa

SEQ ID NO: 4 - protein, 435 aa, Bacillus altitudinis
MSGFTALSEAELNDLYAARQKEYETYKSKNLHLDMSRGKPSPKQLDLSMGMLDVVTSKDAMTAEDGTDVRNYGGLTGLPETKKFFASVLNLKPEQIIIGGNSSLNMMHDTIARAMTHGVYGSKTPWGELPKVKFLAPSPGYDRHFAICEHFNIEMITVDMKSDGPDMDQVEKLVAEDEAIKGIWCVPKYSNPDGITYSDEVVDRLASMQTKADDFRIFWDDAYAVHHLTDTPDTLKDIFQAVDKAGHANRVFMFASTSKITFPGSGVALMASSQDNVSFIQKQLSVQTIGPDKINQLRHLRFFKNPEGLKEHMKKHAAIIKPKFDLVLSILDEKLGGTGIAEWHKPNGGYFISLNTLDHCAKAVVQKAKEAGVTLTGAGATYPYGNDPLDRNIRIAPTFPTLEELEQAIDIFTLCVQLVSIEKLLSEKSQSAPTV

SEQ ID NO: 5 - DNA, 867 bp, Unknown
  Obtained from environmental sample
catatggacgctctgggttattacaatggcaactggggtccgctggatgaaatgacggttccgatgaatgatcgtggctgttactttggtgacggcgtgtatgatgcgacctgcgccgtcaacggtgtgatctttgcgctggatgaacatattgaccgtttctttaactctgccaaactgctggaaatcaatattagtctgaccaaggaagaactgaaaaagacgctgaacgaaatgtactctaaggtggataagggcgaatatctggtttactggcaggtcacccgcggcacgggtcgtcgctcgcatgtttttccggcgggtccgagcaacctgtggattatcattaaaccgaaccacatcgataatctgtaccgtaaaatcaagctgattaccatggatgacacgcgcttcctgcactgcaatattaaaaccctgaacctgatcccgaatgtcattgcatcccagcgtgcactggaagctggctgccatgaagctgtctttcaccgcggtgaaaccgtgacggaatgtgcgcattcaaacgttcacatcattaaaaatggccgtttcatcacccatccggctgataacctgattctgcgtggtacggcacgctcacacctgctgcaagcttgtgtgcgtctgaatatcccggttgacgaacgcgaattttccctgtcagaactgttcgatgccgacgaagtgctggttagctctagtggcaccctgggtctgtcggcagaagaaattgatggcaaaaaggcgggcggtaaagccccggaactgctgaaaaagatccaagacgaagttctgcgtgaattcattgaagccaccggttacacgccggaatggagccgcgtcctcgag

SEQ ID NO: 6 - protein, 286 aa, Unknown
  Obtained from environmental sample
MDALGYYNGNWGPLDEMTVPMNDRGCYFGDGVYDATCAVNGVIFALDEHIDRFFNSAKLLEINISLTKEELKKTLNEMYSKVDKGEYLVYWQVTRGTGRRSHVFPAGPSNLWIIIKPNHIDNLYRKIKLITMDDTRFLHCNIKTLNLIPNVIASQRALEAGCHEAVFHRGETVTECAHSNVHIIKNGRFITHPADNLILRGTARSHLLQACVRLNIPVDEREFSLSELFDADEVLVSSSGTLGLSAEEIDGKKAGGKAPELLKKIQDEVLREFIEATGYTPEWSRV

(19) United States
(12) Patent Application Publication
(10) Pub. No.: US 2013/0164792 A1
(43) Pub. Date: Jun. 27, 2013

(54) METHOD FOR PRODUCING CRYSTALS OF MUTIVALENT METAL SALT OF (2R, 4R) MONATIN

(71) Applicant: Ajinomoto Co., Inc., Tokyo (JP)

(72) Inventors: Kenichi MORI, Kawasaki-shi (JP); Masakazu Sugiyama, Kawasaki-shi (JP); Mika Moriya, Kawasaki-shi (JP); Shunichi Suzuki, Kawasaki-shi (JP); Hidemi Fujii, Kawasaki-shi (JP); Kyohei Fujiwara, Kawasaki-shi (JP)

(73) Assignee: Ajinomoto Co., Inc., Tokyo (JP)

(21) Appl. No.: 13/678,909

(22) Filed: Nov. 16, 2012

Related U.S. Application Data
(60) Provisional application No. 61/560,964, filed on Nov. 17, 2011.

(51) Int. Cl. C07D 209/20; C12P 17/10

(52) U.S. Cl. CPC C07D 209/20; C12P 17/10
USPC 435/106; 548/502

(57) ABSTRACT

The present invention provides a method of efficiently producing a crystal of a (2R,4R)Monatin multivalent metal salt that has a good sweetness property and is excellent in storage stability. Specifically, the present invention provides the method of producing the crystal of the (2R,4R)Monatin multivalent metal salt comprising allowing an aldehyde or one or two or more enzymes capable of forming (2R,4R)Monatin from (2S,4R)Monatin to be acted on an aqueous solution containing the (2S,4R)Monatin in the presence of a multivalent metal ion to obtain the crystal of the (2R,4R)Monatin multivalent metal salt. The aldehyde may preferably be an aromatic aldehyde. The one or two or more enzyme may preferably be a racemase or one or more aminotransferases. The multivalent metal may preferably be a bivalent alkaline earth metal.

### CROSS-REFERENCE TO RELATED APPLICATION

This application claims benefits of the priority of U.S. Patent Provisional Application No. 61/560,964 filed on Nov. 17, 2011, the entire contents of which are incorporated herein by reference.

### TECHNICAL FIELD

The present invention relates to a method of producing a crystal of a (2R,4R)Monatin multivalent metal salt.

### BACKGROUND ART

(2S,4S) isomer of 4-hydroxy-4-(3-indolylmethyl)-2-aminoglutaric acid (hereinafter also referred to as “Monatin”) is known to be contained in barks of Schlerochitom ilicifolius, a plant that grows naturally in Northern Transvaal in South Africa, have a sweetness that is several hundreds times sweeter than sucrose, and be an amino acid derivative useful as a sweetener (Patent Literature 1).

Various methods of producing the Monatin have been reported (Non-Patent Literatures 1 to 3, Patent Literatures 2 and 3). Some methods of producing optically active Monatin have been studied, but many steps are required for the production with a less yield, and thus the methods have not been always suitable industrially.

Patent Literature 4 discloses a method of obtaining a crystal by epimerizing (2R,4R)Monatin from (2S,4R)Monatin. However, the obtained crystal is a crystal of a (2R,4R)Monatin potassium salt, and thus, its stability is not always satisfied depending on its formulation.

### PRIOR ART DOCUMENTS

### Patent Literatures

Patent Literature 1: JP 64-25757-A
Patent Literature 2: U.S. Pat. No. 5,994,559
Patent Literature 3: International Publication WO03/059865 Pamphlet
Patent Literature 4: U.S. Pat. No. 7,396,941

### Non-Patent Literatures

Non-Patent Literature 1: Tetrahedron Letters, 2001, Vol. 42, No. 39, p. 6793-6796
Non-Patent Literature 2: Organic Letters, 2000, Vol. 2, No. 19, p. 2967-2970
Non-Patent Literature 3: Synthetic Communication, 1994, Vol. 24, No. 22, p. 3197-3211.

### DISCLOSURE OF INVENTION

### Problem to be Solved by the Invention

It is an object of the present invention to provide a method of efficiently producing a crystal of a (2R,4R)Monatin multivalent metal salt that has a good sweetness property and is excellent in storage stability.

### Means for Solving Problem

As a result of an extensive study, the present inventors have found that the above problem can be accomplished by allowing an aldehyde or one or two or more enzymes capable of forming (2R,4R)Monatin from (2S,4R)Monatin to be acted on an aqueous solution containing the (2S,4R)Monatin in the presence of a multivalent metal ion, and have completed the present invention.

Accordingly, the present invention includes the followings.

[1] A method of producing a crystal of a (2R,4R)Monatin multivalent metal salt, comprising contacting (2S,4R)Monatin with an aldehyde or one or two or more enzymes capable of forming (2R,4R)Monatin from the (2S,4R)Monatin in an aqueous solution containing a multivalent metal ion to precipitate the crystal of the (2R,4R)Monatin multivalent metal salt.
[2] A method of producing a crystal of a (2R,4R)Monatin multivalent metal salt, comprising allowing an aldehyde or one or two or more selected from the group consisting of racemases and aminotransferases to be acted on an aqueous solution containing (2S,4R)Monatin in the presence of a multivalent metal ion to obtain the crystal of the (2R,4R)Monatin multivalent metal salt.
[3] The method of producing the crystal of the (2R,4R)Monatin multivalent metal salt according to [1] or [2], wherein the aldehyde is an aromatic aldehyde.
[4] The method of producing the crystal of the (2R,4R)Monatin multivalent metal salt according to [1], wherein the enzyme capable of forming the (2R,4R)Monatin from the (2S,4R)Monatin is a racemase.
[5] The method of producing the crystal of the (2R,4R)Monatin multivalent metal salt according to [4], wherein the racemase comprises the amino acid sequence of SEQ ID NO:2.
[6] The method of producing the crystal of the (2R,4R)Monatin multivalent metal salt according to [1], wherein the enzyme capable of forming the (2R,4R)Monatin from the (2S,4R)Monatin is an aminotransferase.
[7] The method of producing the crystal of the (2R,4R)Monatin multivalent metal salt according to [1], wherein the enzyme capable of forming the (2R,4R)Monatin from the (2S,4R)Monatin is an L-amino acid aminotransferase and a D-amino acid aminotransferase.
[8] The method of producing the crystal of the (2R,4R)Monatin multivalent metal salt according to [1], wherein the enzyme capable of forming the (2R,4R)Monatin from the (2S,4R)Monatin is an L-amino acid aminotransferase, a D-amino acid aminotransferase, and a racemase.
[9] The method of producing the crystal of the (2R,4R)Monatin multivalent metal salt according to [7] or [8], wherein the L-amino acid aminotransferase comprises the amino acid sequence of SEQ ID NO:4 and the D-amino acid aminotransferase comprises the amino acid sequence of SEQ ID NO:6.
[10] The method of producing the crystal of the (2R,4R)Monatin multivalent metal salt according to any of [1] to [9], wherein the multivalent metal is a bivalent alkaline earth metal.
[11] The method of producing the crystal of the (2R,4R)Monatin multivalent metal salt according to [10], wherein the alkaline earth metal is magnesium.
[12] The method of producing the crystal of the (2R,4R)Monatin multivalent metal salt according to [1] to [11], wherein a pH value of the aqueous solution is 4 to 11.
[13] The method of producing the crystal of the (2R,4R)Monatin multivalent metal salt according to any of [1] to [12], wherein an organic solvent in an amount of 5% by volume or less is present in the aqueous solution.
[14] The method of producing the crystal of the (2R,4R)Monatin multivalent metal salt according to any of [1] to [13], wherein the crystal has characteristic X ray diffraction peaks at 8.9°, 11.2°, 15.0°, 17.8°, and 22.5° as diffraction angles (2θ±0.2°, CuKα).
[15] The method of producing the crystal of the (2R,4R)Monatin multivalent metal salt according to any of [1] to [13], wherein the crystal has characteristic X ray diffraction peaks at 4.9°, 16.8°, 18.0°, and 24.6° as diffraction angles (2θ±0.2°, CuKα).
[16] The method of producing the crystal of the (2R,4R)Monatin multivalent metal salt according to any of [1] to [15], comprising collecting the crystal of the (2R,4R)Monatin multivalent metal salt.
[17] The method of producing the crystal of the (2R,4R)Monatin multivalent metal salt according to any of [1] to [16], wherein an amount of the formed (2R,4R)Monatin is allowed to be increased by simultaneously performing an isomerization from the (2S,4R)Monatin to the (2R,4R)Monatin and a crystallization of the (2R,4R)Monatin multivalent metal salt.
[18] The method of producing the crystal of the (2R,4R)Monatin multivalent metal salt according to any of [1] to [17], further comprising facilitating the precipitation of the crystal of the (2R,4R)Monatin multivalent metal salt by adding an organic solvent to the aqueous solution after forming the (2R,4R)Monatin.

### Effect of the Invention

The present invention can provide the method of efficiently producing the (2R,4R)Monatin multivalent metal salt that has the good sweetness property and is excellent in storage stability, by allowing the aldehyde or one or two or more enzymes capable of forming the (2R,4R)Monatin from the (2S,4R)Monatin to be acted on the aqueous solution containing the (2S,4R)Monatin in the presence of the multivalent metal ion.

### BRIEF DESCRIPTION OF DRAWINGS

FIG. 1 is a view illustrating an isomerization from (2S,4R)Monatin to (2R,4R)Monatin by a racemase.

FIG. 2 is a view illustrating a formation of the (2R, 4R)Monatin from the (2S,4R)Monatin via 4R-IHOG by one aminotransferase. The aminotransferase does not have a stereoselectivity at position 2, and thus can reversibly form 4R-IHOG from the (2S,4R)Monatin followed by forming the (2R,4R)Monatin from 4R-IHOG. 4R-IHOG: 4R-(indole-3-yl-methyl)-4-hydroxy-2-oxoglutaric acid.

FIG. 3 is a view illustrating the formation of the (2R, 4R)Monatin from the (2S,4R)Monatin via 4R-IHOG by two aminotransferases. The first aminotransferase forms 4R-IHOG from the (2S,4R)Monatin and then the second aminotransferase forms the (2R,4R)Monatin from 4R-IHOG.

FIG. 4 is a view illustrating the formation of the (2R, 4R)Monatin from the (2S,4R)Monatin via 4R-IHOG by two aminotransferases (L-amino acid aminotransferase and D-amino acid aminotransferase). The L-amino acid aminotransferase forms 4R-IHOG from the (2S,4R)Monatin and then the D-amino acid aminotransferase forms the (2R, 4R)Monatin from 4R-IHOG. By adding a keto acid (or an L-amino acid or a D-amino acid) and the racemase capable of converting the L-amino acid into the D-amino acid to a reaction system, it is possible to couple a side reaction by the L-amino acid aminotransferase (keto acid→L-amino acid) and a side reaction by the D-amino acid aminotransferase (D-amino acid→keto acid).

FIG. 5 is a view illustrating a preferable example of the reaction shown in FIG. 4. By adding PA (or L-Ala or D-Ala) and an alanine racemase to a reaction system, it is possible to couple a side reaction by the L-amino acid aminotransferase (PA→L-Ala) and a side reaction by the D-amino acid aminotransferase (D-Ala→PA). PA: pyruvic acid; Ala: alanine.

FIG. 6 is a view illustrating a preferable example of the reaction shown in FIG. 4. By adding α-KG (or L-Glu or D-Glu) and a glutamic acid racemase to a reaction system, it is possible to couple a side reaction by the L-amino acid aminotransferase (α-KG→L-Glu) and a side reaction by the D-amino acid aminotransferase (D-Glu→α-KG). α-KG: α-ketoglutaric acid; Glu: glutamic acid.

FIG. 7 is a view showing characteristic X ray diffraction peaks of a crystal of a ((2R,4R)Monatin)₂ magnesium salt.

FIG. 8 is a graph showing a concentration change of the (2S,4R)Monatin and the (2R,4R)Monatin over time in a reaction solution. SR-Monatin: (2S,4R)Monatin; RR-Monatin: (2R,4R)Monatin (hereinafter the same meaning will apply).

FIG. 9 is a graph showing a concentration change of the (2S,4R)Monatin and the (2R,4R)Monatin over time in a supernatant of a reaction solution after adding a magnesium salt.

FIG. 10 is a view of graphs showing a concentration change of the (2S,4R)Monatin and the (2R,4R)Monatin over time in a reaction solution. Upper graphs show the concentration change of the (2S,4R)Monatin (left) and the (2R,4R)Monatin (right) over time in a reaction solution under a condition with or without a preferential crystallization. Middle graphs show the concentration change of the (2S,4R)Monatin (left) and the (2R,4R)Monatin (right) over time in a whole reaction solution or in a supernatant in the case of only enzyme reaction (without a preferential crystallization). Lower graphs show the concentration change of the (2S,4R)Monatin (left) and the (2R,4R)Monatin (right) over time in a whole reaction solution or in a supernatant when a enzymatic reaction and a preferential crystallization were carried out simultaneously.

FIG. 11 is a view showing characteristic X ray diffraction peaks of a crystal of a ((2R,4R)Monatin)₂ magnesium salt obtained in Example 11.

FIG. 12 is a view showing characteristic X ray diffraction peaks of a crystal of a ((2R,4R)Monatin)₂ magnesium salt obtained in Reference Example 3.

FIG. 13 is a view showing characteristic X ray diffraction peaks of a crystal of a ((2R,4R)Monatin)₂ magnesium salt obtained in Reference Example 4.

FIG. 14 is a view showing characteristic X ray diffraction peaks of a crystal of a ((2R,4R)Monatin)₂ magnesium salt obtained in Reference Example 5.

### BEST MODES FOR CARRYING OUT THE INVENTION

The present invention provides a method of producing a crystal of a (2R,4R)Monatin multivalent metal salt. The method of the present invention comprises contacting (2S, 4R)Monatin with an aldehyde or one or two or more enzymes capable of forming (2R,4R)Monatin from the (2S,4R)Monatin in an aqueous solution containing a multivalent metal ion to precipitate the crystal of the (2R,4R)Monatin multivalent metal salt. In other word, it is a method comprising allowing an aldehyde or one or two or more enzymes capable of forming (2R,4R)Monatin from (2S, 4R)Monatin to be acted on an aqueous solution containing the (2S,4R)Monatin in the presence of a multivalent metal ion to obtain the crystal of the (2R,4R)Monatin multivalent metal salt.

In the light of reaction process, the method of the present invention comprises (a) isomerizing the (2S,4R)Monatin to form the (2R,4R)Monatin (isomerization) and (b) contacting the formed (2R,4R)Monatin with the multivalent metal ion to precipitate the crystal of the (2R, 4R)Monatin multivalent metal salt (crystallization). The method of the present invention may also comprise collecting the obtained crystal. The steps (a) and (b) can be performed separately or simultaneously (in other words, in parallel), but it is preferable to perform them simultaneously. By performing the steps (a) and (b) simultaneously, it is possible to increase an amount of the formed (2R,4R)Monatin. When the steps (a) and (b) are performed separately, macromolecules may be removed (e.g., centrifugation, filtration) from the reaction solution obtained in the step (a) in order to avoid crystallization inhibition by the macromolecules. An addition of a seed crystal into the solution may be included upon crystallization. The precipitation of the crystal of the (2R,4R)Monatin multivalent metal salt can also be facilitated by adding an organic solvent to the aqueous solution after forming the (2R,4R)Monatin. A wet crystal can be obtained easily by subjecting the crystal precipitated in the step (b) to a separation step such as a filtration step. The obtained wet crystal may be washed. A dry crystal can be obtained by drying the wet crystal. These procedures will be described in detail later.

The (2S,4R)Monatin used in the present invention can be obtained by a known method, and for example, those produced by a chemical synthesis method or an enzymatic method can be used. Any method can be used as the chemical synthesis method as long as the (2S,4R)Monatin is formed. For example, the (2S,4R)Monatin may be obtained by crystallizing a solution of reduced (4R)-4-hydroxy-4-(3-indolylmethyl)-2-hydroxyiminoglutamic acid using the method described in U.S. Pat. No. 7,064,219 and Patent Document 4. As the enzymatic method, any methods can be used as long as the (2S,4R)Monatin is formed, and for example, the (2S, 4R)Monatin may be obtained by enzymatically yielding (4R)-IHOG followed by oximation/reduction, column purification, and crystallization in combination of the methods described in UP Patents No. 7,297,800 and 7,064,219. The (2S,4R)Monatin that is a material in the method of the present invention may be a material containing the (2S,4R)Monatin at least in a small amount, and may be in any form of a crystal, powder and solution (aqueous solution, organic solution, water/organic solvent mixed solution). Therefore, the purified (2S,4R)Monatin may be used, or a reaction solution containing the (2S,4R)Monatin [reaction solution used for producing (2S,4R)Monatin)] may be used as the aqueous solution containing the (2S,4R)Monatin for the method of the present invention.

The (2S,4R)Monatin in a form of a salt, a hydrate, or a hydrate of a salt may also be provided in the aqueous solution used in the method of the present invention. The salt may include salts of monovalent metals (e.g., potassium and sodium) and non-metals (e.g., ammonium), and salts of multivalent metals (e.g., bivalent metals and trivalent metals) described later. Examples of the hydrate may include monohydrate, dihydrate, trihydrate, tetrahydrate, pentahydrate, hexahydrate, heptahydrate, octahydrate, and nonahydrate.

An aldehyde used for the present invention will be described in detail. The aldehyde may include an aliphatic aldehyde and an aromatic aldehyde, and the aromatic aldehyde is preferable.

As the aliphatic aldehyde, for example, a saturated or unsaturated aldehyde having 1 to 7 carbon atoms such as formaldehyde, acetaldehyde, propionaldehyde, n-butylaldehyde, 1-butylaldehyde, n-valeraldehyde, capronaldehyde, n-heptylaldehyde, acrolein, and methacrolein can be used.

As the aromatic aldehyde, for example, benzaldehyde, salicylaldehyde, m-hydroxybenzaldehyde, p-hydroxybenzaldehyde, o-nitrobenzaldehyde, p-nitrobenzaldehyde, 5-nitrosalicylaldehyde, 3,5-dichlorosalicylaldehyde, anisaldehyde, o-vanillin, vanillin, furfural, pyridoxal, pyridoxal 5-phosphate and the like can be used. Pyridoxal, 5-nitrosalicylaldehyde, and 3,5-dichlorosalicylaldehyde are particularly preferable as the aromatic aldehyde.

The aldehyde can be used in the range of 0.01 to 1 molar equivalent and more preferably 0.05 to 0.5 molar equivalents relative to an amount of the Monatin present in a system.

Next, the enzyme capable of forming the (2R,4R)Monatin from the (2S,4R)Monatin used for the present invention will be described in detail. Examples of the enzyme capable of forming the (2R,4R)Monatin from the (2S, 4R)Monatin may include a racemase, an aminotransferase, an amino acid dehydrogenase and the like, and the racemase and the aminotransferase are preferable. These may be used alone in combination of two or more.

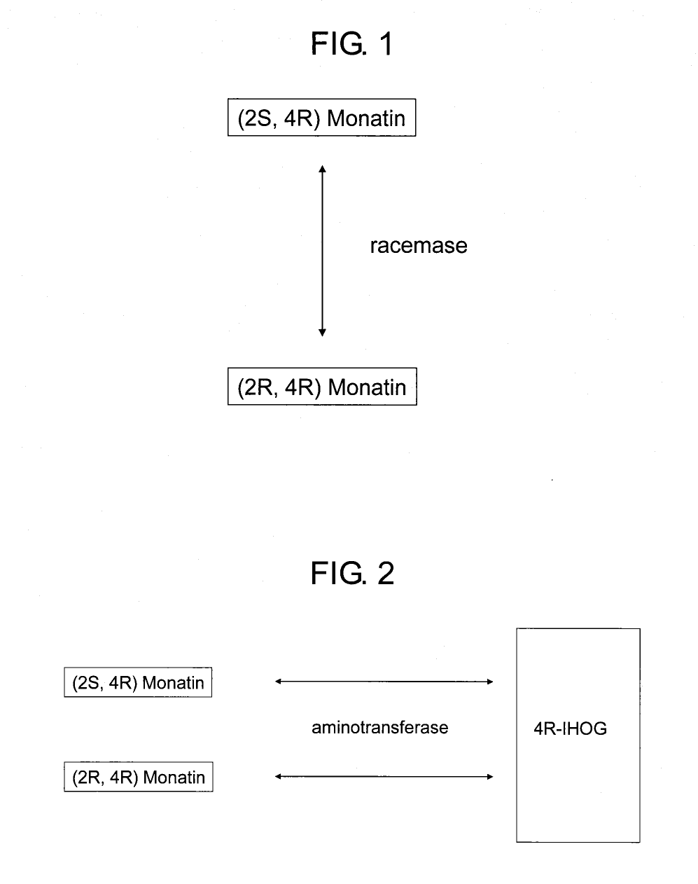

The racemase used for the present invention will be described in detail (FIG. 1). The racemase is not particularly limited as long as it is the enzyme having an activity to convert the (2S,4R)Monatin into the (2R,4R)Monatin. The enzyme having such an activity may also be referred to as an epimerase. In the present invention, as long as the enzyme having another name has such an activity, the enzyme is referred to as the racemase and included within the scope of the present invention. A person skilled in the art can appropriately obtain the racemase having such an activity. For example, a person in the art can obtain the racemase having such an activity by using a certain screening method. Such a screening method may include reacting a reaction solution containing 20 mM (2S, 4R)Monatin [or (2R,4R)Monatin], 100 mM Tris-HCl (pH 8.0), 50 μM pyridoxal phosphate, and an enzyme solution (purified enzyme, crude enzyme, or the like) at 30° C. for 16 hours. A protein specified by the amino acid sequence of SEQ ID NO:2 is a racemase obtained by such a screening method.

A protein comprising an amino acid sequence exhibiting a significant amino acid identity to the amino acid sequence of SEQ ID NO:2 and having an activity to convert the (2S,4R)Monatin to the (2R,4R)Monatin can be used as the racemase. Examples of the amino acid sequence exhibiting the significant amino acid identity to the amino acid sequence of SEQ ID NO:2 may include amino acid sequences exhibiting 70% or more, preferably 80% or more, more preferably 85% or more, still more preferably 90% or more, particularly preferably 95% or more, 96% or more, 97% or more, 98% or more, or 99% or more amino acid identity to the amino acid sequence of SEQ ID NO:2.

The identity between the amino acid sequences can be determined using algorithm BLAST by Karlin and Altschul (Pro. Natl. Acad. Sci. USA, 90, 5873 (1993)) or FASTA by Pearson (Methods Enzymol., 183, 63 (1990)). A program called BLASTP has been developed based on this algorithm BLAST (see http://www.ncbi.nlm.nih.gov). Thus, the identity between the amino acid sequences may be calculated using this program with default setting. In addition, for example, a numerical value obtained by calculating a percentage of matching counts with setting of unit size to compare=2 using full length polypeptide chains encoded in ORFs and using software GENETYX Ver. 7.0.9 from Genetyx Corporation may be used as the identity between the amino acid sequences. The lowest value in the values derived from these calculations may be employed as the identity between the amino acid sequences.

The racemase may also be (a) a protein comprising the amino acid sequence of SEQ ID NO:2 or (b) a protein comprising an amino acid sequence having one or several amino acid mutations (e.g., substitutions, deletions, additions, or insertions) in the amino acid sequence of SEQ ID NO:2 and having an activity to convert the (2S,4R)Monatin into the (2R,4R)Monatin. One or several amino acid mutations may be introduced into one region or multiple different regions in the amino acid sequence. The term “one or several” indicates a range in which a three-dimensional structure and an activity of a protein are not greatly impaired. The term “one or several” in the case of the protein refers to, for example, 1 to 150, preferably 1 to 100, more preferably 1 to 50, 1 to 30, 1 to 20, 1 to 10, or 1 to 5. Such a mutation may be attributed to a naturally occurring mutation based on an individual difference and a species difference in organisms (e.g., microorganisms, animals) (mutant or variant) carrying a gene encoding the racemase.

Positions of amino acid residues, at which the mutations are to be introduced, are obvious to a person skilled in the art. Specifically, a person skilled in the art can recognize a correlation between structure and function, since a person skilled in the art 1) can compare amino acid sequences from multiple proteins having the same kind of activity (e.g., the amino acid sequence of ID NO:2 and amino acid sequences of other racemases), 2) clarify regions that are relatively conserved and regions that are not relatively conserved, and then 3) predict regions capable of playing an important role in function and a region incapable of playing the important role in function e from the regions that are relatively conserved and the regions that are not relatively conserved. Therefore, a person skilled in the art can specify the positions of the amino acid residues at which the mutations are to be introduced in the amino acid sequence of the racemase.

When an amino acid residue is mutated by substitution, the substitution of the amino acid may be conservative substitution. The term “conservative substitution” refers to the substitution of a certain amino acid with an amino acid having a similar side chain. Families of amino acid residues having the similar side chain are well-known in the art. Examples of such families may include amino acids having a basic side chain (e.g., lysine, arginine, and histidine), amino acids having an acidic side chain (e.g., aspartic acid and glutamic acid), amino acids having a non-charged polar side chain (e.g., asparagine, glutamine, serine, threonine, tyrosine, and cysteine), amino acids having a non-polar side chain (e.g., glycine, alanine, valine, leucine, isoleucine, proline, phenylalanine, methionine, and tryptophan), amino acids having a branched side chain at β position (e.g., threonine, valine, and isoleucine), amino acids having an aromatic side chain (e.g., tyrosine, phenylalanine, tryptophan, and histidine), amino acids having a side chain containing a hydroxyl group (e.g., alcohol or phenolic) (e.g., serine, threonine, and tyrosine), and amino acids having a sulfur-containing side chain (e.g., cysteine and methionine). Preferably, the conservative substitution between the amino acids may be the substitution between aspartic acid and glutamic acid, the substitution among arginine, lysine and histidine, the substitution between tryptophan and phenylalanine, the substitution between phenylalanine and valine, the substitution among leucine, isoleucine and alanine, and the substitution between glycine and alanine.

The racemase is preferably a protein that is encoded by DNA hybridized under a stringent condition with the nucleotide sequence complementary to the nucleotide sequence of SEQ ID NO:1 and has an activity to convert the (2S,4R)Monatin into the (2R,4R)Monatin. The “stringent condition” refers to a condition where a so-called specific hybrid is formed whereas a nonspecific hybrid is not formed. Under a condition where polynucleotides having the identity are hybridized each other whereas polynucleotides having the identity lower than that are not hybridized, the identity of the polynucleotide each other is preferably 70% or more, more preferably 80% or more, still more preferably 90% or more, especially more preferably 95% or more, and particularly preferably 98% or more. Specifically, such a condition may include hybridization in 6×SSC (sodium chloride/sodium citrate) at about 45° C. followed by one or two or more washings in 0.2×SSC and 0.1% SDS at 50 to 65° C.

A tag for purification may be added to an N terminus or a C terminus of the racemase used in the present invention. Utilization of the tag for the purification can make the purification of the racemase convenient. Examples of the tag for the purification may include a histidine (His) tag, a calmodulin binding peptide (CBP), a strep-tag II, and FLAG.

The racemase can be obtained from a racemase-producing microorganism or by the synthesis in a cell-free system. Examples of the racemase-producing microorganism may include microorganisms that naturally produce the racemase and transformants that express the racemase. The racemase can be used as a purified enzyme, a crude enzyme, or an immobilized enzyme.

When the transformant that expresses the racemase is used as the racemase-producing microorganism, this transformant can be made by making an expression vector for the racemase and then introducing this expression vector into a host. As the host for expressing the racemase, for example, various prokaryotic cells from bacteria belonging to genus Escherichia such as Escherichia coli, bacteria belonging to genus Corynebacterium (e.g., Corynebacterium glutamicum) and bacteria belonging to genus Bacillus (e.g., Bacillus subtilis), and eukaryotic cells from microorganisms belonging to genus Saccharomyces (e.g., Saccharomyces cerevisiae), genus Pichia (e.g., Pichia stipitis), and genus Aspergillus (e.g., Aspergillus oryzae) can be used. The example of the preferable host is E. coli. When E. coli is used as the host, it is also more preferable to use an expression vector in which a polynucleotide encoded by the DNA sequence of SEQ ID NO:1 is inserted.

For a reaction condition for an isomerization reaction, a person skilled in the art can establish a suitable condition by a simple preliminary experiment. A concentration of the racemase is preferably 0.001 to 1000 U/mL and more preferably 0.1 to 100 U/mL based on an amount of the Monatin present in a reaction system (1 U represents an activity to isomerize 1 μmol of the Monatin for one minute). A pH value in the reaction system in the isomerization reaction is preferably pH 5.0 to 11.0, more preferably pH 6.0 to 10.0, and still more preferably pH 7.0 to 9.0. The concentration of the (2S,4R)Monatin [or (2R, 4R)Monatin] to be added to the reaction system is preferably 10 mM to 3.0 M and more preferably 100 mM to 1.0 M. In this case, the Monatin may be added to the reaction system before starting the reaction, but may be added intermittently or continuously after starting the reaction. A reaction temperature is not particularly limited as long as the isomerization reaction progresses and is preferably 15° C. to 60° C. and more preferably 25° C. to 42° C. A reaction time period is preferably 1 to 120 hours and more preferably 1 to 24 hours. Pyridoxal phosphate (PLP) is not necessarily required to be added to the reaction system, but the concentration of PLP when added is not particularly limited as long as the isomerization reaction progresses, and is preferably 10 to 100 μM. A salt such as NaCl or KCl may be added in order to stabilize the racemase in a reaction solution for the racemase or a liquid such as buffer used for purification or dialysis of the racemase. When the racemase is purified or dialyzed, it is preferable to add 50 mM to 500 mM NaCl or KCl to an objective solution.

When a crystal of a ((2R,4R)Monatin)₂ magnesium salt is precipitated from the reaction solution containing the (2R,4R)Monatin and the (2S,4R)Monatin with which the racemase was reacted, a person skilled in the art can establish the suitable condition by a simple preliminary experiment. The concentration of the (2R,4R)Monatin in the reaction solution is preferably 20 mM to 3 m and more preferably 50 mM to 1 M. A magnesium source added to the reaction solution is preferably 10 mM to 3 M and more preferably 25 mM to 1 M. The temperature is preferably 0 to 60° C. and more preferably 10 to 40° C. The time period is preferably 3 hours to one week and more preferably 24 to 60 hours. A centrifuged supernatant of the reaction solution or a filtrate from an ultrafiltration of the reaction solution may be used, or an amount of a seed crystal to be added may be increased in order to avoid inhibition of the crystallization by macromolecules.

Next, the aminotransferase used in the present invention will be described in detail. Those having both an activity to convert 4R-IHOG into the (2S,4R)Monatin (2S stereoselective activity) and an activity to convert 4R-IHOG into the (2R,4R)Monatin (2R stereoselective activity) can be used as the aminotransferase. The converting activity of the aminotransferase may be reversible. The enzyme having the both activity may be used in an unpurified state. The enzyme having each activity may be purified separately and then combined for the use. In the present invention, the enzyme having another name is referred to as the aminotransferase and included in the present invention as long as the enzyme has such an activity.

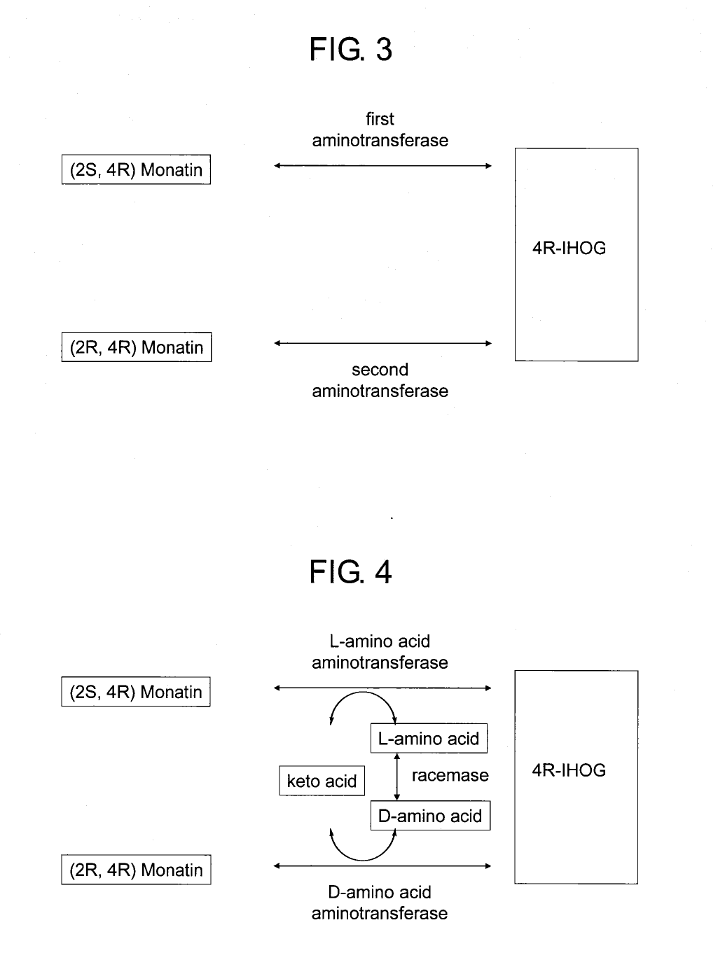

The aminotransferase will be further described based on FIGS. 2 and 3. As shown in FIG. 2, the aminotransferase may convert the (2S,4R)Monatin into the (2R,4R)Monatin via 4R-IHOG. Alternatively, as shown in FIG. 3, the first aminotransferase may convert (2S,4R)Monatin into 4R-IHOG and the second aminotransferase may convert 4R-IHOG into the (2R,4R)Monatin.

A person skilled in the art can appropriately acquire such an aminotransferase. A wild type or mutant aminotransferase is known to be able to isomerize a given compound by a reversible aminotransferase reaction via another intermediate compound. In such a case, the aminotransferase has an apparent racemase activity. For example, an aspartic acid aminotransferase is known to be able to acquire the apparent racemase activity (e.g., see Kochhar, Sunil, et al., European Journal of Biochemistry (1992), 203 (3), 563-9). Therefore, the aminotransferase that can act upon the (2S,4R)Monatin to exert the apparent racemase activity can be used in the present invention. The amount of the aminotransferase to be added to the reaction system is not particularly limited as long as the reaction can proceed appropriately.

Examples of the aminotransferase having the activity to convert 4R-IHOG into the (2S,4R)Monatin (2S stereoselective activity) (first aminotransferase) may include L-amino acid aminotransferases, and L-aspartic acid aminotransferases. Specifically, examples of such an aminotransferase may include enzymes described in WO2012/050125.

Examples of the aminotransferase having the activity to convert 4R-IHOG into the (2R,4R)Monatin (2R stereoselective activity) (second aminotransferase) may include D-amino acid aminotransferases. Examples of the D-amino acid aminotransferase may include the enzymes described in WO03/056026, WO2009/088949, and WO2012/147674.

It is preferable that the first aminotransferase be the L-amino acid aminotransferase and the second aminotransferase be the D-amino acid aminotransferase (FIG. 4). The amounts of the first and second aminotransferases to be added to the reaction system are not particularly limited as long as the reaction can proceed appropriately.

A protein comprising an amino acid sequence exhibiting the significant amino acid identity to the amino acid sequence of SEQ ID NO:4 and having an activity to convert 4R-IHOG into the (2S,4R)Monatin can be used as the L-amino acid aminotransferase. Examples of the amino acid sequence exhibiting the significant amino acid identity to the amino acid sequence of SEQ ID NO:4 may include amino acid sequences exhibiting 70% or more, preferably 80% or more, more preferably 85% or more, still more preferably 90% or more, particularly preferably 95% or more, 96% or more, 97% or more, 98% or more, or 99% or more amino acid identity to the amino acid sequence of SEQ ID NO:4.

A protein comprising an amino acid sequence exhibiting the significant amino acid identity to the amino acid sequence of SEQ ID NO:6 and having an activity to convert 4R-IHOG into the (2R,4R)Monatin can be used as the D-amino acid aminotransferase. Examples of the amino acid sequence exhibiting the significant amino acid identity to the amino acid sequence of SEQ ID NO:6 may include amino acid sequences exhibiting 70% or more, preferably 80% or more, more preferably 85% or more, still more preferably 90% or more, particularly preferably 95% or more, 96% or more, 97% or more, 98% or more, or 99% or more amino acid identity to the amino acid sequence of SEQ ID NO:6. The amino acid identity can be determined by the aforementioned method.

The L-amino acid aminotransferase may also be (a′) a protein comprising the amino acid sequence of SEQ ID NO:4 or (b′) a protein comprising an amino acid sequence having one or several amino acid residue mutations (e.g., substitutions, deletions, additions, or insertions) in the amino acid sequence of SEQ ID NO:4, and having an activity to convert 4R-IHOG into the (2S,4R)Monatin.

The D-amino acid aminotransferase may also be (a) a protein comprising the amino acid sequence of SEQ ID NO:6 or (b) a protein comprising an amino acid sequence having one or several amino acid residue mutations (e.g., substitutions, deletions, additions, or insertions) in the amino acid sequence of SEQ ID NO:6, and having an activity to convert 4R-IHOG into the (2R,4R)Monatin.

The mutations of one or several amino acid residue may be introduced into one region or multiple different regions in the amino acid sequence. The term “one or several” is the same as described above. The positions of the amino acid residues, at which the mutations are to be introduced in the amino acid sequence, are obvious to a person skilled in the art as described above. When the amino acid residue is mutated by the substitution, the substitution of the amino acid residue may be the conservative substitution as described above.

The L-amino acid aminotransferase is preferably a protein that is encoded by DNA hybridized under a stringent condition with the nucleotide sequence complementary to the nucleotide sequence of SEQ ID NO:3 and has an activity to convert 4R-IHOG into the (2S,4R)Monatin. This activity of the L-amino acid aminotransferase can be reversible.

The D-amino acid aminotransferase is preferably a protein that is encoded by DNA hybridized under a stringent condition with the nucleotide sequence complementary to the nucleotide sequence of SEQ ID NO:5 and has an activity to convert 4R-IHOG into the (2R,4R)Monatin. This activity of the D-amino acid aminotransferase can be reversible.

The stringent condition is as described above.

The tag for the purification may be added to the N terminus or the C terminus of the aminotransferase used in the present invention. The utilization of the tag for the purification can make the purification of the aminotransferase convenient. Examples of the tag for the purification may include the histidine (His) tag, the calmodulin binding peptide (CBP), the strep-tag II, and FLAG.

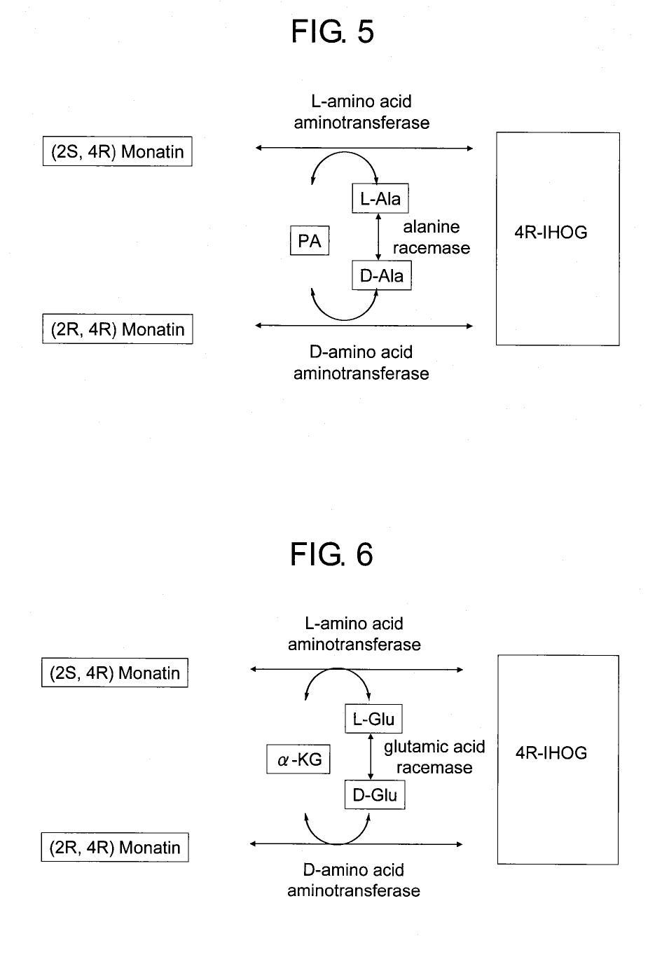

When both the L-amino acid aminotransferase and the D-amino acid aminotransferase are used, it is possible to couple the side reaction by the L-amino acid aminotransferase (keto acid→L-amino acid) and the side reaction by the D-amino acid aminotransferase (D-amino acid→keto acid) (FIG. 4) by adding a keto acid (or an L-amino acid or a D-amino acid) and the racemase capable of converting the L-amino acid into the D-amino acid in a small amount to the reaction system. Examples of the “amino acid” in the L-amino acid or the D-amino acid may include alanine, glutamic acid, asparagine, cysteine, glutamine, isoleucine, leucine, methionine, phenylalanine, proline, serine, threonine, tryptophan, tyrosine, valine, aspartic acid, arginine, histidine, and lysine, which are naturally occurring α-amino acids. The keto acid is a keto acid formed from the aforementioned L-amino acid or D-amino acid via an action of the amino acid aminotransferase. Since various racemases capable of converting the L-amino acid into the D-amino acid are known, such racemases can be used in the present invention. Preferably, the keto acid is pyruvic acid, the L-amino acid is L-alanine, and the D-amino acid is D-alanine, when the alanine racemase is used as the racemase (FIG. 5). In addition, the keto acid is α-ketoglutaric acid, the L-amino acid is L-glutamic acid and the D-amino acid is D-glutamic acid, when the glutamic acid racemase is used as the racemase (FIG. 6). The concentration of the keto acid (e.g., pyruvic acid, or α-ketoglutaric acid), the concentration of the L-amino acid (L-alanine, or L-glutamic acid), the concentration of the D-amino acid (D-alanine, or D-glutamic acid) to be added to the reaction system, and the amount of the racemase (e.g., alanine racemase, or glutamic acid racemase) to be added to the reaction system are not particularly limited as long as the reaction can proceed appropriately.

When the aminotransferase is allowed to be acted on the (2S,4R)Monatin to isomerize the (2S,4R)Monatin to the (2R,4R)Monatin, a person skilled in the art can establish a suitable condition by a simple preliminary experiment. The concentration of the (2S,4R)Monatin to be added to the reaction system is preferably 10 mM to 3.0 M and more preferably 300 mM to 1.0 M. In this case, the Monatin may be added to the reaction system before starting the reaction and may be added intermittently or continuously after starting the reaction. The reaction pH value is preferably pH 5.0 to 11.0, more preferably pH 6.0 to 10.0, and still more preferably pH 7.0 to 9.0. The reaction temperature is not particularly limited as long as the aminotransferase reaction proceeds, and is preferably 10° C. to 60° C. and more preferably 15° C. to 42° C. The reaction time period is not particularly limited, and is preferably 1 to 180 hours and more preferably 1 to 76 hours. Pyridoxal phosphate (PLP) is not necessarily added to the reaction system, but when PLP is added, the concentration of PLP is not particularly limited as long as the aminotransferase reaction proceeds, and is preferably 10 to 100 μM. Both the L-amino acid aminotransferase and the D-amino acid aminotransferase may be used as the aminotransferase. In this case, the concentration of each aminotransferase is not particularly limited as long as the reaction proceeds, and is preferably 0.01 to 10 mg/mL and more preferably 0.1 to 5 mg/L. The keto acid (or L-amino acid or D-amino acid) and the racemase capable of converting the L-amino acid into the D-amino acid may be added in a small amount to the reaction system. In this case, the concentration of the keto acid is preferably 1 to 100 mM and more preferably 10 to 50 mM. The concentration of the D-amino acid is preferably 1 to 100 mM and more preferably 20 to 50 mM. The concentration of the racemase is not particularly limited as long as the reaction progresses, and is preferably 0.1 to 100 μg/mL and more preferably 1 to 10 μg/mL.

When a crystal of a ((2R,4R)Monatin)₂ magnesium salt is precipitated from a reaction solution containing the (2S, 4R)Monatin and the (2R,4R)Monatin in which the aminotransferase is allowed to be acted on the (2S,4R)Monatin, a person skilled in the art can establish a suitable condition by a simple preliminary experiment. The concentration of the (2R,4R)Monatin in the reaction solution is preferably 20 mM to 3 M and more preferably 50 mM to 1 M. The amount of a magnesium source to be added to the reaction system may be sufficient for the amount of the Monatin present in the reaction system, and is preferably 10 mM to 1.5 M and more preferably 25 mM to 500 mM. The reaction temperature is preferably 0 to 60° C. and more preferably 10 to 40° C. The reaction time period is preferably 3 hours to one week and more preferably 24 hours to 60 hours. A centrifuged supernatant of the reaction solution or a filtrate from the ultrafiltration of the reaction solution may be used, or the amount of the seed crystal to be added may be increased in order to avoid crystallization inhibition by macromolecules.

When the racemase or the aminotransferase is allowed to be acted on the (2S,4R)Monatin to form the (2R,4R)Monatin from the (2S,4R)Monatin, the reaction with the enzyme alone comes to a certain equilibrium state in the aqueous solution. However, if the reaction with enzyme proceeds simultaneously with the crystallization of the (2R, 4R)Monatin multivalent metal salt, the (2R,4R)Monatin can be removed as a salt crystal out of the reaction system. Thus, the equilibrium state shifts toward the formation of the (2R,4R)Monatin, and the amount of the formed (2R,4R)Monatin can be increased. Also when the (2R,4R)Monatin is formed from the (2S,4R)Monatin by action of an aldehyde catalyst, the (2R,4R)Monatin can be removed as the salt crystal out of the reaction system by progressing the isomerization reaction simultaneously with the crystallization of the (2R,4R)Monatin multivalent metal salt. Thus, the equilibrium state shifts toward the formation of the (2R,4R)Monatin, and the amount of the formed (2R,4R)Monatin can be increased.

When a crystal of a (2R,4R)Monatin multivalent metal salt is precipitated simultaneously while the (2S,4R)Monatin is isomerized to the (2R,4R)Monatin by action of the aminotransferase, a person skilled in the art can establish a suitable condition by a simple preliminary experiment. The concentration of the (2S,4R)Monatin to be added to the reaction system is preferably 50 mM to 3 M and more preferably 100 mM to 1 M. In this case, the Monatin may be added to the reaction system before starting the reaction, or may be added intermittently or continuously after starting the reaction. The reaction pH value is preferably pH 5.0 to 10.0, and more preferably pH 6.0 to 7.0 to inhibit a retroaldol degradation. The reaction temperature is not particularly limited as long as the aminotransferase reaction progresses, and is preferably 10° C. to 60° C. and more preferably 15° C. to 42° C. The reaction time period is not particularly limited, and is preferably 10 to 240 hours and more preferably 120 to 240 hours. Pyridoxal phosphate (PLP) is not necessarily required to be added to the reaction system, but the concentration of PLP when added is not particularly limited as long as the aminotransferase reaction progresses, and is preferably 10 to 100 μM. Both the L-amino acid aminotransferase and the D-amino acid aminotransferase may be used as the aminotransferase. In this case, the concentration of each aminotransferase is not particularly limited as long as the reaction progresses, and is preferably 0.01 to 10 mg/mL and more preferably 0.1 to 10 mg/mL. The keto acid (or L-amino acid or D-amino acid) and the racemase capable of converting the L-amino acid into the D-amino acid may be added in a small amount to the reaction system. In this case, the concentration of the keto acid is preferably 1 to 100 mM and more preferably 10 to 50 mM. The concentration of the D-amino acid is preferably 1 to 100 mM and more preferably 20 to 50 mM. The concentration of the racemase is not particularly limited as long as the reaction progresses, and is preferably 0.1 to 100 μg/mL and more preferably 1 to 10 μg/mL. In this case, the aminotransferase or the racemase may be added to the reaction system before starting the reaction, but may be added intermittently or continuously after starting the reaction in order to compensate inactivation and inhibition during the reaction. The amount of the magnesium source to be added to the reaction system may be sufficient for the amount of the Monatin present in the reaction system, and for example is preferably 25 mM to 1.5 M and more preferably 50 mM to 500 mM. In this case, the magnesium source may be added to the reaction system before starting the reaction, but may be added intermittently or continuously after starting the reaction in order to compensate the inactivation and the inhibition during the reaction. In order to avoid inhibition of the crystallization by the enzyme, an enzyme source may be isolated in a dialysis membrane or immobilized. In order to prevent inhibition of the crystallization by oxides in the reaction solution, an inert gas such as an argon gas or a nitrogen gas may be blown into the reaction solution. To prevent inhibition of the crystallization, the amount of the seed crystal may be increased.

When the (2R,4R)Monatin is formed from the (2S,4R)Monatin by the enzymatic isomerization reaction, the reaction with the enzyme alone comes to the certain equilibrium state in the aqueous solution. However, in the present method, the (2R,4R)Monatin multivalent metal salt can be removed out of the reaction system before the isomerization comes to the equilibrium state by progressing the enzymatic isomerization reaction simultaneously with the preferential crystallization of the (2R,4R)Monatin multivalent metal salt by the multivalent metal ion. Thus, it is possible to increase the amount of the (2R,4R)Monatin multivalent metal salt capable of being formed by the enzymatic isomerization reaction.

The aminotransferase can be obtained from an aminotransferase-producing microorganism or by synthesis in the cell-free system. Examples of the aminotransferase-producing microorganism may include microorganisms that naturally produce the aminotransferase and transformants that express the aminotransferase. The aminotransferase can be used as a purified enzyme, a crude enzyme, or an immobilized enzyme.

When the transformant that expresses the aminotransferase is used as the aminotransferase-producing microorganism, this transformant can be made by making an expression vector for the aminotransferase and then introducing this expression vector into a host. For example, the prokaryotic cell or the eukaryotic cell as described above can be used as the host for expressing the aminotransferase. An example of the preferable host is E. coli.

In the method of the present invention, an aqueous solution is used as a solvent, and examples thereof may include water and buffers containing no organic solvent, and water and buffers containing the organic solvent in a small amount. Examples of the buffer may include a Tris buffer, a phosphate buffer, a carbonate buffer, a borate buffer, and an acetate buffer. As the organic solvent, an organic solvent miscible with the water is used, and alcohols such as methanol, ethanol, propanol, and isopropanol are particularly preferable. Different two or more organic solvents may be used in mixture. A percentage of the organic solvent in the aqueous solution is preferably 10% by volume or less, more preferably 5% by volume or less, and still more preferably 2% by volume or less. When the aldehyde is used as the catalyst, the aqueous solution containing the organic solvent (e.g., aldehyde alone) in a small amount may be used as the reaction solution. On the other hand, when the enzyme is used as the catalyst, the water or the buffer containing no organic solvent may be used as the reaction solution.

The multivalent metal ion used for the present invention is not particularly limited, and can be formed by adding a multivalent metal salt into an aqueous solution. The multivalent metal salt added to the reaction solution for forming the multivalent metal ion is not particularly limited as long as the metal is an element having bivalence or more in a periodic table and can be formed a salt with the Monatin. The metal, ingestion of which is acceptable in human is preferable. Specifically, examples of a bivalent metal salt may include alkaline earth metal salts such as salts with magnesium, calcium, strontium, and barium, and transition metal salts such as salts with iron, nickel, copper, and zinc. Examples of a trivalent metal salt may include metal salts with aluminium and the like. These salts may be used alone or in combination of two or more. Among them, the bivalent metal salt is preferable, the alkaline earth metal salt is more preferable, a magnesium salt, a calcium salt, a strontium salt, and a barium salt are still preferable, the magnesium salt, the calcium salt, and the barium salt are still more preferable, and the magnesium salt and the calcium salt are particularly preferable. The multivalent metal salt may be a non-hydrate or a hydrate (e.g., monohydrate, dihydrate, trihydrate, tetrahydrate, pentahydrate, hexahydrate, heptahydrate, octahydrate or nonahydrate).

Examples of a convenient method of obtaining the multivalent metal salt used in the present invention may include methods of treating an inorganic multivalent metal compound such as calcium hydroxide, magnesium hydroxide, calcium carbonate, or magnesium carbonate, and an organic multivalent metal compound such as calcium acetate, magnesium acetate, calcium oxalate, magnesium nitrate, calcium lactate, or magnesium lactate by various methods such as neutralization and salt exchange.

The amount of the multivalent metal ion may be sufficient for the amount of the Monatin present in the system, and for example, the multivalent metal ion can be used in the range of 0.4 to 0.6 molar equivalents and more preferably 0.45 to 0.55 molar equivalents.

The temperature in the method of the present invention is preferably 0 to 50° C. and more preferably 25 to 40° C. The time period in the method of the present invention is preferably 5 hours to one week and more preferably 10 hours to 48 hours.

The pH value in the method of the present invention is preferably 4 to 10 and more preferably 5 to 9 and still more preferably 6 to 8. The pH value can be adjusted using an acid or an alkaline. The acid to be used is not particularly limited, and an organic acid such as an acetic acid and an inorganic acid such as a hydrochloric acid and a sulfuric acid can be used. The alkaline is not particularly limited, and an alkali metal hydroxide such as sodium hydroxide and potassium hydroxide, and an organic base such as ammonia and amine can be used.

The precipitation of a crystal of a (2R,4R)Monatin multivalent metal salt can also be facilitated by adding the organic solvent to the aqueous solution after forming the (2R,4R)Monatin. It is also preferable that the organic solvent be added after the amount of the (2R,4R)Monatin dissolved in the aqueous solution is saturated. The organic solvent to be added to the aqueous solution for facilitating the precipitation of the crystal of the (2R, 4R)Monatin multivalent metal salt is not particularly limited as long as the organic solvent is miscible with water, and examples thereof may include methanol, ethanol, n-propanol, isopropanol, n-butanol, t-butanol, sec-butanol, propylene glycol, acetonitrile, and THF.

The crystal of the (2R,4R)Monatin multivalent metal salt can be obtained according to the method of the present invention. Among them, a crystal of a ((2R,4R)Monatin)₂ bivalent metal salt is preferable, a crystal of a ((2R,4R)Monatin)₂ alkaline earth metal salt is more preferable, a crystal of a ((2R,4R)Monatin)₂ magnesium salt, a crystal of a ((2R,4R)Monatin)₂ calcium salt, a crystal of a ((2R, 4R)Monatin)₂ strontium salt, and a crystal of a ((2R,4R)Monatin)₂ barium salt are still preferable, the crystal of the ((2R,4R)Monatin)₂ magnesium salt, the crystal of the ((2R,4R)Monatin)₂ calcium salt, and the crystal of the ((2R,4R)Monatin)₂ barium salt are still more preferable, and the crystal of the ((2R,4R)Monatin)₂ magnesium salt and the crystal of the ((2R,4R)Monatin)₂ calcium salt are particularly preferable because their ingestion is acceptable in the human and they are easily prepared.

The crystal of the ((2R,4R)Monatin)₂ magnesium salt will be described among the crystal of the (2R,4R)Monatin multivalent metal salts in the present invention.

The precipitation of the crystal can be achieved by leaving to stand the aqueous solution containing the (2R, 4R)Monatin and the magnesium source or subjecting it to the crystallization with stirring, by the method described above. The concentration of the crystal of the (2R,4R)Monatin in the solvent is not particularly limited as long as the Monatin is supersaturated and the crystal is precipitated, and is preferably 0.1 to 60% by weight. The concentration is more preferably 1 to 50% by weight and still more preferably 5 to 45% by weight in terms of achieving a viscosity of the solution suitable for the production. The temperature at which the crystal is dissolved is not particularly limited as long as the crystal continues to dissolve, and is preferably 15 to 40° C.

A wet crystal can be obtained easily by subjecting the precipitated crystal to a separation step such as a filtration step. Crystal washing is not particularly limited as long as crystal solvent exchange is not caused, and water can be used. The solvent used for the crystal washing may contain solvents such as methanol, ethanol, n-propanol, isopropanol, n-butanol, t-butanol, sec-butanol, propylene glycol, acetonitrile, THF, acetone and DMF which can be miscible with the water, inorganic salts and the like as long as the crystal solvent exchange is not caused.

The wet crystal thus obtained can be led to a dry crystal by subjecting to a known drying step. A drying equipment used for the drying step is not particularly limited, a temperature zone in which the ((2R,4R)Monatin)₂ magnesium salt is not dissolved can be used, and drying under reduced pressure or gas flow drying, hot wind drying and the like can be used.

The crystal of the ((2R,4R)Monatin)₂ magnesium salt obtained in the method of the present invention has characteristic X ray diffraction peaks at 8.9°, 11.2°, 15.0°, 17.8°, and 22.5°, or 4.9°, 16.8°, 18.0°, and 24.6° as diffraction angles (2θ±0.2°, CuKα).

The crystal of the (2R,4R)Monatin multivalent metal salt obtained in the method of the present invention can further be purified by combining known separation purification procedures such as concentration, concentration under reduced pressure, extraction with solvent, crystallization, recrystallization, solvent exchange, treatment with activated charcoal, chromatography using an ion exchange resin, a synthetic adsorption resin or the like, if necessary.

The crystal of the (2R,4R)Monatin multivalent metal salt obtained by the method of the present invention can be converted into another salt such as an alkali metal salt such as a potassium salt, a sodium salt, or a calcium salt or an ammonium salt, or free form.

A method known to a person skilled in the art can be used as a method of converting the (2R,4R)Monatin multivalent metal salt into the free form or the other salt. Examples of the method of obtaining the free form may include a method of dissolving or suspending the (2R,4R)Monatin multivalent metal salt in water, alcohol, or a mixed solvent thereof and neutralizing the solution or the suspension with an acid such as a hydrochloric acid or a sulfuric acid followed by precipitating a crystal of the free form of the (2R,4R)Monatin, and a method of dissolving the salt in water, and decomposing/desalting via a strong acidic ion exchange resin to separate the free form in an eluant followed by distillating off the solvent or precipitating a crystal of the free form, and the like.

The methods of converting the (2R,4R)Monatin multivalent metal salt into the other salt can include a method of dissolving the crystal of the free form obtained above in an alkaline metal aqueous solution such as a solution of sodium hydroxide, potassium hydroxide or calcium hydroxide or an ammonia aqueous solution or the like followed by distilling off the solvent or precipitating a crystal of the (2R,4R)Monatin salt, and a method of adding the alkali metal aqueous solution such as the solution of sodium hydroxide, potassium hydroxide or calcium hydroxide or the ammonia aqueous solution or the like into a resin eluate containing the free form of Monatin followed by distilling off the solvent or precipitating a crystal of the (2R,4R)Monatin salt, and the like.

In Addition, various food materials as well as various additives that can be used as oral products such as beverages, food products, pharmaceutical products, quasi drugs, and feedstuffs can be used to an extent that the effect of the present invention is not prevented.

The crystal of the Monatin multivalent metal salt in the present invention can be used for the oral products such as the beverages, the food products, the pharmaceutical products, the quasi drugs, and the feedstuffs. A dosage form thereof is not particularly limited, and examples thereof may include powders, granules, cubes, pastes, and liquids.

### EXAMPLES

The present invention will be described in detail with reference to the following Examples, but the present invention is not limited to these Examples.

### (HPLC Analysis Condition)

In Example 1, an HPLC analysis was carried out under a condition shown in this Example.

Detector: Ultraviolet spectrophotometer (measurement wavelength: 210 nm)
Column temperature: 40° C.
Column: CAPCELLPACK C18 Type MGII, internal diameter: 3 mm, length: 25 cm, particle diameter: 5 μm, Shiseido Co., Ltd.
Mobile phase:

Solution A: 20 mM potassium dihydrogen phosphate aqueous solution:acetonitrile=100:5

Solution B: 20 mM potassium dihydrogen phosphate aqueous solution:acetonitrile=60:40

Gradient program: see following Table 1.

TABLE 1
Gradient program

| Time (minutes) | Mobile phase A (%) | Mobile phase B (%) |
|---|---|---|
| 0.0 | 100 | 0 |
| 15.0 | 100 | 0 |
| 40.0 | 0 | 100 |
| 45.0 | 0 | 100 |
| 45.1 | 100 | 0 |

Flow: 0.45 mL/minute
Injection amount: 10 μL
Analysis time period: 60 minutes

Contents of water and magnesium in the obtained crystal [((2R,4R)Monatin)₂ magnesium salt] were analyzed by a method of measuring a water content and a cation analysis method by ion chromatography. The performed method of measuring the water content and the cation analysis method are shown in detail below.

### (Method of Measuring Water Content)

Measurement apparatus: Hiranuma Automatic Water Content Measurement Apparatus AQV-2000 (manufactured by Hiranuma Sangyo Corporation)

Measurement condition: Titration liquid=Hydranal Composite 5K (manufactured by Riedel de Haen)

(Cation analysis method)

Apparatus: Tosoh IC2001

Column: TSKgel SuperIC-Cation (4.6×150 mm)

Guard column: TSKgel SuperIC-Cation (1 cm)

Suppress gel: TSKgel TSKsuppressIC-C

Column temperature: 40° C.

Eluant flow: 0.7 mL/minute

Sample injection amount: 30 μL

Detection: Electric conductivity

Eluant composition: 2.2 mM methanesulfonic acid+1.0 mM 18-crown-6-ether+0.5 mM histidine mixed aqueous solution

### [Powder X Ray Diffraction Measurement Method]

1) 0.5 g of a sample crystal was collected and ground in an agate mortar for 60 seconds. The obtained powder was set on a glass plate and flattened by pressurizing from above. This was immediately set in a powder X ray diffraction apparatus, and measured under the following condition.

2) The measurement of powder X ray diffraction by Cu-Kα ray was carried out using an X ray diffraction apparatus PW3050 manufactured by Spectris Co., Ltd., under the condition of X ray tube: Cu, tube current: 30 mA, tube voltage: 40 kV, sampling width: 0.020°, scanning rate: 3°/minute, wavelength: 1.54056 angstroms, measured diffraction angle range (2θ): 4 to 30°.

Measurement program: X'PERT DATA COLLECTION
Analysis program: X'PERT High Score

3) Obtained data were processed into a graph using

Excel, and characteristic sharp maximum peaks in the range of 4 to 30° were read out. An error of the diffraction angle in this method is ±0.2°

### Reference Example 1

### Synthesis of (2S,2R)Monatin

(2R,4R)Monatin potassium salt monohydrate was preferentially crystallized by adding 149.00 g of ethanol to 101.40 g of a concentrated solution for reductive reaction (containing 36.62 g (125.28 mmol) of Monatin, (2S, 4R):(2R,4R)=32:68) followed by adding 0.25 g of the (2R, 4R) monatin potassium salt monohydrate as a seed crystal, and stirring at 56° C. for 4 hours. The precipitated crystal was separated by filtration (wet crystal 31.27 g) to obtain 225.80 g of a mother solution (containing 22.41 g (76.68 mmol) of Monatin, (2S,4R):(2R,4R)=53:47). This mother solution was cooled to 10° C. and then stirred for 5 hours to precipitate a crystal of (2S,4R)Monatin potassium salt dihydrate. The precipitated crystal was separated by filtration (wet crystal 32.74 g), and dried under reduced pressure to obtain 9.88 g (15.68 mmol) of the objective (2S, 4R)Monatin potassium salt dihydrate (HPLC purity: 55.5%). 9.35 g of this crude crystal was dissolved in 25.37 g of water, 58.99 g of ethanol was added thereto, and the mixture was stirred at 25° C. for 5 hours to precipitate a purified crystal of the (2S,4R)Monatin potassium salt dihydrate. The precipitated crystal was separated by filtration (wet crystal 4.49 g), and dried under reduced pressure to obtain 3.75 g (9.62 mmol) of the objective (2S, 4R)Monatin potassium salt dihydrate (HPLC purity: 96.0%).

The above manipulation was repeated again to obtain the (2S,4R)Monatin potassium salt dihydrate with 100% HPLC purity.

### Reference Example 2

### Measurement of Solubility of Monatin Metal Salt

Solubility of a (2S,4R)Monatin potassium salt and a (2R,4R)Monatin potassium salt in water (H₂O) was measured. 1 g of the (2S,4R)Monatin potassium salt or 1 g of the (2R,4R)Monatin potassium salt was added to 1 g of the water, the mixture was stirred at 25° C., a slurry was filtrated out, and the resulting filtrate was analyzed by HPLC. As a result, the solubility of the (2S,4R)Monatin potassium salt and the (2R,4R)Monatin potassium salt was 20.1% by weight and 37.8% by weight, respectively. The solubility of the (2R,4R)Monatin potassium salt was higher than the solubility of the (2S,4R)Monatin potassium salt.

Then, Solubility of a (2S,4R)Monatin magnesium salt and a (2R,4R)Monatin magnesium salt in the water was measured. The solubility was measured in the same manner as above. For the (2S,4R)Monatin magnesium salt, firstly, the same amount of magnesium chloride was added to the (2S, 4R)Monatin potassium salt, and then ethanol was further added thereto. Then, the resulting slurry was filtrated and the wet crystal was dried under reduced pressure to prepare the (2S,4R)Monatin magnesium salt. For the (2R, 4R)Monatin magnesium salt, firstly, the same amount of magnesium chloride was added to the (2R,4R)Monatin potassium salt, and then methanol was further added thereto. Then, the resulting slurry was filtrated and the wet crystal was dried under reduced pressure to prepare the (2R, 4R)Monatin magnesium salt.

As a result, the solubility of the (2S,4R)Monatin magnesium salt and the (2R,4R)Monatin magnesium salt was 14.5% by weight and 1.6% by weight, respectively. The solubility of the (2R,4R)Monatin magnesium salt was much lower than the solubility of the (2S,4R)Monatin magnesium salt.

### Reference Example 3

### Preparation of Crystal of ((2R,4R)Monatin)₂ Magnesium Salt

10 g (28.5 mmol) of a crystal of a (2R,4R)Monatin potassium salt was dissolved in 20 mL of water, and 28.3 mL of an aqueous solution of 501 mM magnesium chloride was added thereto at room temperature. After stirring at 25° C. for 18 hours, 80 g of methanol was added to the Monatin solution, and then stirred at room temperature for 6 hours. A precipitated crystal was filtered off, the slurry was washed with 50 g of 80% methanol for 2 hours, filtered, and then dried under reduced pressure at 40° C. The dried crystal was stored in an incubator at temperature of 44° C. and at humidity of 78% for 24 hours, and further dried under reduced pressure at 40° C. to yield 8.8 g of a crystal of a ((2R,4R)Monatin)₂ magnesium salt.

¹H-NMR (in D₂O)

### 1.94-2.01 (1H, q), 2.57-2.61 (1H, q), 2.99-3.03 (1H, d), 3.19-3.23 (1H, d), 3.54-3.57 (1H, q), 7.05-7.17 (3H, m), 7.40-7.42 (1H, m), 7.64-7.66 (1H, m)

ESI-MS: 293.1 (M+H)⁺, 291.1 (M−H)⁻

Water content: 10.8% by weight
Magnesium content: 3.6% by weight
Characteristic X ray diffraction peaks (2θ±0.2°, CuKα): 8.9°, 11.2°, 15.0°, 17.8°, and 22.5° (FIG. 12)

### Reference Example 4

### Preparation of Crystal of ((2R,4R)Monatin)₂ Magnesium Salt

120 g (345 mmol) of a crystal of a (2R,4R)Monatin potassium salt was dissolved in 150 mL of water, and 4.15 g (34.5 mmol) of magnesium sulfate was added thereto at 60° C. An aqueous solution (water 100 mL) of 16.61 g (138 mmol) of magnesium sulfate was further added over 6.4 hours. After the addition, a precipitated crystal was filtered off, and washed with 100 mL of water to obtain a wet crystal (204.7 g). The wet crystal was dried under reduced pressure at 40° C. to obtain 105 g of a crystal of a magnesium salt. To further remove potassium sulfate contaminated in a small amount, 400 mL of water was added to 105 g of the dried crystal, and then stirred at 25° C. for 1.5 hours. The resulting slurry was filtered off, and washed with 300 mL of water to obtain a wet crystal (153.9 g). The wet crystal was dried under reduced pressure at 40° C. to yield 85.7 g of a crystal of a ((2R,4R)Monatin)₂ magnesium salt.

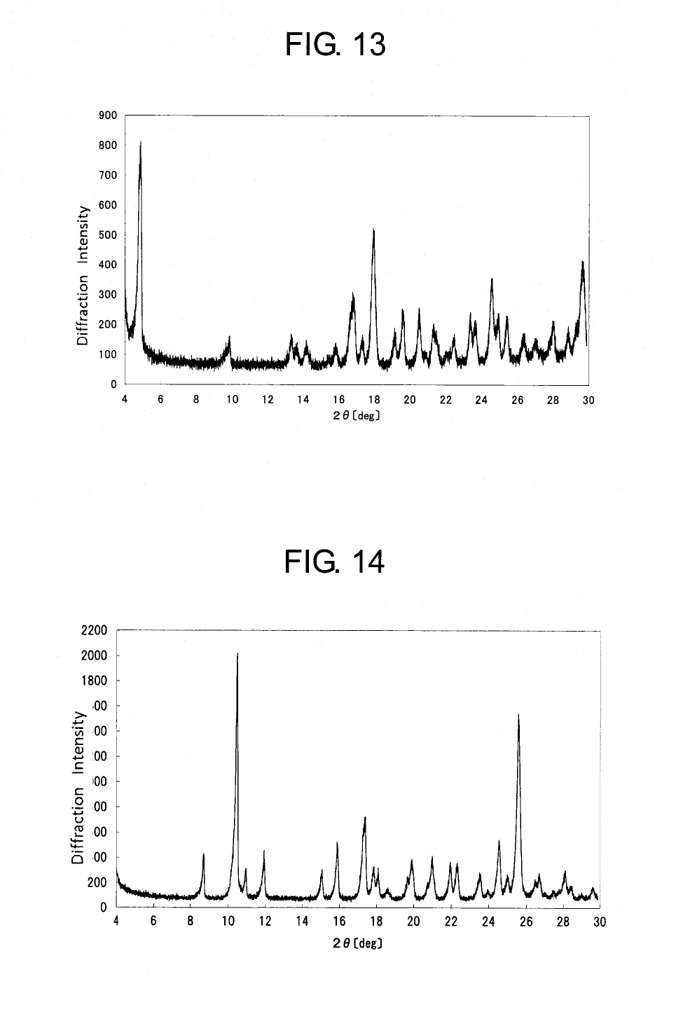

Characteristic X ray diffraction peaks (2θ±0.2°, CuKα): 4.9°, 16.8°, 18.0°, and 24.6° (FIG. 13).
Water content: 6.0% by weight
Magnesium content: 3.61% by weight

### Reference Example 5

### Preparation of Crystal of ((2R,4R)Monatin)₂ Magnesium Salt

30 g (100 mmol) of a crystal of a free form of (2R, 4R)Monatin was dispersed in 300 mL of water, and 3.21 g (55 mmol) of magnesium hydroxide was added thereto at 65° C. After stirring at 65° C. for one hour, a precipitated crystal (27.28 g) was filtered off and dried under reduced pressure at 40° C. for 4 hours to yield 22.29 g of a crystal of a magnesium salt.

Water content: 21.22% by weight
Magnesium content: 3.45% by weight
Characteristic X ray diffraction peaks (2θ±0.2°, CuKα): 8.7°, 10.5°, 15.9°, 17.4°, 21.0° and 25.6° (FIG. 14).

### Example 1

### Synthesis of (2R,4R)Monatin

1.25 g (3.42 mmol) of the (2S,4R)Monatin potassium salt dihydrate was dissolved in 5 mL of water, and 0.05216 g (0.0428 mmol) of salicylaldehyde and 0.3476 g (1.71 mmol) of magnesium chloride hexahydrate were added. After stirring at 65° C. for 42 hours, a seed crystal [the crystal of the ((2R,4R)Monatin)₂ magnesium salt having the same crystal form as that of the crystal obtained in Reference Example 3] was added, and the mixture was further stirred for 191 hours. The resulting slurry solution was cooled to room temperature, then filtrated, a crystal was washed with 1 g of water, and the wet crystal was dried at 40° C. under reduced pressure to yield 0.446 g of (2R,4R)Monatin. The resulting crystal, mother solution, and washing solution were analyzed by HPLC to determine its yield and analyze its quality. From characteristic X ray diffraction peaks, it was confirmed that the obtained crystal of the ((2R,4R)Monatin)₂ magnesium salt had the same crystal form as that of the crystal obtained in Reference Example 3.

HPLC area purity (210 nm): 94.2%

¹H-NMR (in D₂O)

1.93-2.00 (1H, dd), 2.57-2.61 (1H, dd), 2.99-3.02 (1H, d), 3.19-3.22 (1H, d), 3.55-3.56 (1H, dd), 7.04-7.15 (3H, m), 7.39-7.41 (1H, m), 7.64-7.66 (1H, d)

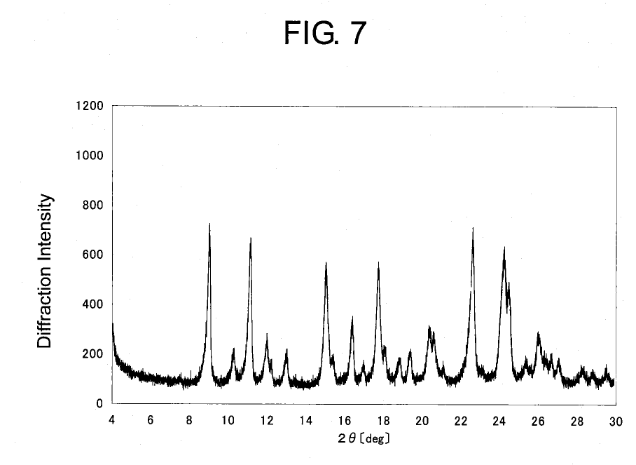

Water content: 14.7% by weight
Magnesium content: 3.4% by weight
Characteristic X ray diffraction peaks (2θ±0.2°, CuKα): 8.9°, 11.2°, 15.0°, 17.8°, and 22.5° (FIG. 7).

### Example 2

### Expression of Racemase in E. Coli

### (1) Construction of Plasmid for Expressing Racemase

A DNA sequence obtained by conferring a NdeI recognition sequence to a 5′ end of a DNA sequence of racemase Rac39 and a XhoI recognition sequence to a 3′ end of the DNA sequence of racemase Rac39 was subjected to Optimum Gene Codon Optimization Analysis supplied from GenScript to design and synthesize a synthesized DNA, a gene expression efficiency of which was optimized in E. coli (SEQ ID NO:1).

The synthesized DNA was treated with restriction enzymes NdeI and XhoI, and ligated to pET-22b (Novagen) also treated with NdeI and XhoI. E. coli was transformed with this ligation solution, and an objective plasmid was selected from ampicillin resistant colonies. This plasmid was designated as pET-22-Rac39-His. Racemase in which a His tag was given to its C terminus (Rac39-His) is expressed in this plasmid.

(2) Purification of His Tag-Added Racemase from E. Coli Expression Clone

E. coli BL21 (DE3) was transformed with the constructed expression plasmid pET-22-Rac39-His to yield a transformant. A loopful of the transformant was inoculated to 100 mL of Overnight Express Instant TB Medium (Novagen) containing 100 mg/L of ampicillin in a 500 mL Sakaguchi flask, and was shaken for 16 hours. Shaking was carried out at 25° C. After the cultivation was completed, microbial cells were collected from 200 mL of the resulting cultured medium by centrifugation, washed with and suspended in 20 mM Tris-HCl (pH 7.6), 300 mM NaCl, and 10 mM imidazole, and sonicated. Cell debris was removed from the disrupted suspension by centrifugation, and the resulting supernatant was used as a soluble fraction.

The obtained soluble fraction was applied onto a His-tag protein purification column His TALON superflow 5 ml Cartridge (Clontech) equilibrated with 20 mM Tris-HCl (pH 7.6), 300 mM NaCl, and 10 mM imidazole, and adsorbed to a carrier. Proteins that had not been adsorbed to the carrier (unadsorbed proteins) were washed out with 20 mM Tris-HCl (pH 7.6), 300 mM NaCl, and 10 mM imidazole, and subsequently adsorbed proteins were eluted with 20 mM Tris-HCl (pH 7.6), 300 mM NaCl, and 150 mM imidazole at a flow rate of 5 mL/minute. The obtained fractions were combined and diluted with 20 mM Tris-HCl (pH 7.6) and 10 μM PLP to use as a racemase solution.

### Example 3

### Isomerization Reaction Using (2S,4R)Monatin or (2R,4R)Monatin as Substrate

A reaction was carried out for 15 minutes using the purified racemase under the following condition. The reaction was carried out in 0.1 mL using an Eppendorf tube. After the reaction was terminated, an equal amount of a reaction stopping solution, 200 mM sodium citrate solution (pH 4.5) was added to the sample. HPLC was used for the analysis.

Reaction condition: 20 mM (2S,4R)Monatin or (2R,4R)Monatin, 50 μM PLP, 100 mM Tris-HCl (pH 8.0), 0.72 mg/mL racemase purified enzyme, 25° C., 120 rpm.

The (2S,4R)Monatin and the (2R,4R)Monatin were quantified by HPLC analysis. An analysis condition is as shown below.

Mobile phase: 20 mM KH₂PO₄/acetonitrile=100/5
Flow rate: 1.0 mL/minute
Column temperature: 40° C.

### Detection: UV 280 nm

### Column: CAPCELL PAK MGII, 4.6×150 mm, 3 μm (Shiseido)

As a result, by the use of Rac39, an activity to form the (2R,4R)Monatin from the (2S,4R)Monatin (0.14 U/mg) and an activity to form the (2S,4R)Monatin from the (2R, 4R)Monatin (0.09 U/mg) were detected (1 U represents the activity to isomerize 1 μmol Monatin per minute). From the above, it was demonstrated that (4R)Monatin could be isomerized at position 2 using the racemase.

### Example 4

### Expression of Amino Acid Aminotransferase in E. Coli

### (1) Construction of Plasmid for Expressing L-Amino Acid Aminotransferase

The NdeI recognition sequence and the XhoI recognition sequence were conferred to the 5′ end of and the 3′ end of a DNA sequence (SEQ ID NO:3) of L-amino acid aminotransferase (AJ1616LAT) obtained from a stored bacterial strain, AJ1616 Bacillus altitudinis.

This DNA sequence was treated with the restriction enzymes NdeI and XhoI, and ligated to pET-22b (Novagen) also treated with NdeI and XhoI. E. coli was transformed with this ligation solution, and an objective plasmid was selected from ampicillin resistant colonies. This plasmid was designated as pET-22-LAT-His. L-amino acid transferase in which the His tag was given to the C terminus (LAT-His) is expressed in this plasmid.

### (2) Construction of Plasmid for Expressing D-Amino Acid Aminotransferase

A DNA sequence obtained by conferring a NdeI recognition sequence to the 5′ end of a DNA sequence of D-amino acid aminotransferase selected by In silico and a XhoI recognition sequence to the 3′ end of the DNA sequence of D-amino acid aminotransferase was subjected to Optimum Gene Codon Optimization Analysis supplied from GenScript to yield a synthesized DNA (SEQ ID NO:5), a gene expression efficiency of which was optimized in E. coli. This synthesized DNA was treated with the restriction enzymes NdeI and XhoI, and ligated to pET-22b (Novagen) also treated with NdeI and XhoI. E. coli was transformed with this ligation solution, and an objective plasmid was selected from ampicillin resistant colonies. This plasmid was designated as pET-22-DAT-His. Racemase in which the His tag was given to the C terminus (DAT-His) is expressed in this plasmid.

(3) Purification of His Tag-Added L-Amino Acid Aminotransferase from E. Coli Expression Clone

E. coli BL21 (DE3) was transformed with the constructed expression plasmid pET-22-LAT-His to yield a transformant. A loopful of the transformant was inoculated to 160 mL of Overnight Express Instant TB Medium (Novagen) containing 100 mg/L of ampicillin in a 500 mL Sakaguchi flask, and was shaken for 16 hours. Shaking was carried out at 37° C. After the cultivation was completed, microbial cells were collected from 800 mL of the resulting cultured medium by centrifugation, washed with and suspended in 20 mM Tris-HCl (pH 7.6), 300 mM NaCl, and 10 mM imidazole, and sonicated. Cell debris was removed from the disrupted suspension by centrifugation, and the resulting supernatant was used as a soluble fraction.

The obtained soluble fraction was applied onto a His-tag protein purification column, His TALON superflow 5 ml Cartridge (Clontech) equilibrated with 20 mM Tris-HCl (pH 7.6), 300 mM NaCl, and 10 mM imidazole, and adsorbed to a carrier. Proteins that had not been adsorbed to the carrier (unadsorbed proteins) were washed out with 20 mM Tris-HCl (pH 7.6), 300 mM NaCl, and 10 mM imidazole, and subsequently adsorbed proteins were eluted with 20 mM Tris-HCl (pH 7.6), 300 mM NaCl, and 150 mM imidazole at a flow rate of 5 mL/minute. Obtained fractions were combined and diluted with 20 mM Tris-HCl (pH 7.6) to use as an L-amino acid aminotransferase solution.

(4) Purification of His Tag-Added D-Amino Acid Aminotransferase from E. Coli Expression Clone

E. coli BL21 (DE3) was transformed with the constructed expression plasmid pET-22-DAT-His to yield a transformant. A loopful of the transformant was inoculated to 160 mL of Overnight Express Instant TB Medium (Novagen) containing 100 mg/L of ampicillin in a 500 mL Sakaguchi flask, and was shaken for 16 hours. Shaking was carried out at 30° C. After the cultivation was completed, microbial cells were collected from 800 mL of the resulting cultured medium by centrifugation, washed with and suspended in 20 mM Tris-HCl (pH 7.6), 300 mM NaCl, and 10 mM imidazole, and sonicated. Cell debris was removed from the disrupted suspension by centrifugation, and the resulting supernatant was used as a soluble fraction.

The obtained soluble fraction was applied onto a His-tag protein purification column, His TALON superflow 5 ml Cartridge (Clontech) equilibrated with 20 mM Tris-HCl (pH 7.6), 300 mM NaCl, and 10 mM imidazole, and adsorbed to the carrier. Proteins that had not been adsorbed to the carrier (unadsorbed proteins) were washed out with 20 mM Tris-HCl (pH 7.6), 300 mM NaCl, and 10 mM imidazole, and subsequently adsorbed proteins were eluted with 20 mM Tris-HCl (pH 7.6), 300 mM NaCl, and 150 mM imidazole at a flow rate of 5 mL/minute. Obtained fractions were combined and diluted with 20 mM Tris-HCl (pH 7.6) to use as a D-amino acid aminotransferase solution.

### Example 5

### Reaction of Forming (2R,4R)Monatin from (2S, 4R)Monatin

A reaction was carried out using the purified L-amino acid aminotransferase and D-amino acid aminotransferase for 76 hours under the following condition. The reaction was carried out in 20 mL with stirring using a 50 mL Falcon tube. 990 μL of TE buffer was added to 10 μL of the sample, the mixture was ultrafiltrated using Amicon Ultra 0.5 mL 10 k (Millipore), and a filtrate was analyzed. HPLC was used for the analysis.

Reaction condition: 300 mM (2S,4R)Monatin, 10 mM pyruvic acid, 20 mM D-Ala, 50·μM PLP, 3 mg/mL L-amino acid aminotransferase solution, 3 mg/mL D-amino acid aminotransferase solution, 0.002 mg/mL alanine racemase solution (Unitika), pH 7 (KOH), 25° C.

The (2S,4R)Monatin and the (2R,4R)Monatin were quantified by the HPLC analysis. The analysis condition is as shown below.

Mobile phase:

A: 20 mM KH₂PO₄/acetonitrile=100/10

B: acetonitrile

Flow rate: 1.0 mL/minute
Column temperature: 40° C.

### Detection: UV 280 nm

Column: CAPCELL PAK C18 TYPE MGII 3 μm, 4.6 mm×150 mm (Shiseido)

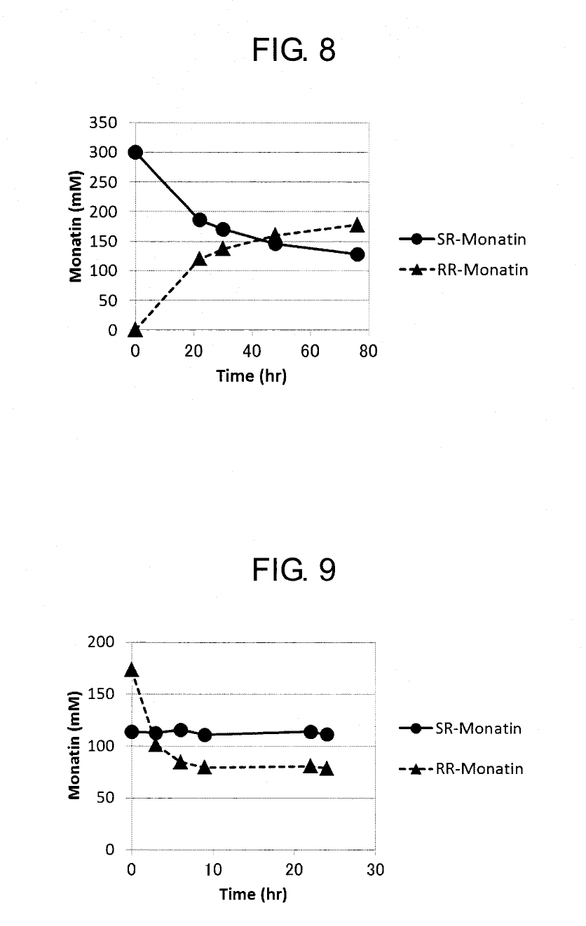

As a result, 128 mM (2S,4R)Monatin and 178 mM (2R, 4R)Monatin were formed after the reaction for 76 hours (FIG. 8). Therefore, it was demonstrated that the (2R,4R)Monatin was formed form the (2S,4R)Monatin using the aminotransferase and the racemase.

### Example 6

### Acquisition of Crystal of ((2R,4R)Monatin)₂ Magnesium Salt from Reaction Solution

150 mM magnesium sulfate heptahydrate was dissolved in the reaction solution described in Example 5, and a supernatant obtained by centrifugation was ultrafiltrated using Amicon Ultra 15 mL 10 k (Millipore). 0.001 g of a seed crystal [the crystal of the ((2R,4R)Monatin)₂ magnesium salt having the same crystal form as that of the crystal obtained in Reference Example 5] was added to the resulting filtrate, and then stirred at 25° C. for 24 hours. The reaction was carried out in 7.5 mL using a 15 mL Falcon tube. 990 μL of TE buffer was added to a sampled reaction solution (10 μL) and a supernatant (10 μL) obtained by centrifuging the reaction solution, the mixture was ultrafiltrated using Amicon Ultra 0.5 mL 10 k (Millipore), and the resulting filtrates were analyzed. HPLC was used for the analysis. HPLC was carried out under the condition described in Example 5.

As a result, the concentration of the (2R,4R)Monatin in the supernatant of the reaction solution was reduced from 174 mM to 81 mM in 24 hours, and a crystal was precipitated (FIG. 9).

The resulting slurry solution was centrifuged, the obtained crystal was washed with water in a small amount, and then the wet crystal was dried at 40° C. under reduced pressure to yield 0.181 g of (2R,4R)Monatin. The obtained crystal, mother solution, and washing solution were analyzed by HPLC to determine its yield and analyze its quality. As a result, it was confirmed that the crystal of the ((2R,4R)Monatin)₂ magnesium salt had been obtained. HPLC was carried out under the condition described above (HPLC analysis condition).

(2R,4R)Monatin content: 73.8% by weight
(2S,4R)Monatin content: 3.27% by weight
Magnesium content: 3.61% by weight
Potassium content: 0.500% by weight

### Example 7

### Simultaneous Performance of (2R,4R)Monatin-Forming Reaction and Preferential Crystallization of ((2R, 4R)Monatin)₂ Magnesium Salt

A reaction was carried out under the following condition using the purified L-amino acid aminotransferase and D-amino acid aminotransferase for 69 hours. The reaction was carried out in 20 mL using a 50 mL falcon tube with shaking at 100 rpm. 990 μL of TE buffer was added to 10 μL of a sample, the mixtures were ultrafiltrated using Amicon Ultra 0.5 mL 10 k (Millipore), and the filtrate was analyzed. HPLC was used for the analysis. HPLC was carried out under the condition described in Example 5.

Reaction condition: 400 mM (2S,4R)Monatin, 10 mM pyruvic acid, 20 mM D-Ala, 50 μM PLP, 5 mg/mL L-amino acid aminotransferase solution, 5 mg/mL D-amino acid aminotransferase solution, 0.0015 mg/mL alanine racemase solution (Unitika), pH 7 (KOH), 25° C.

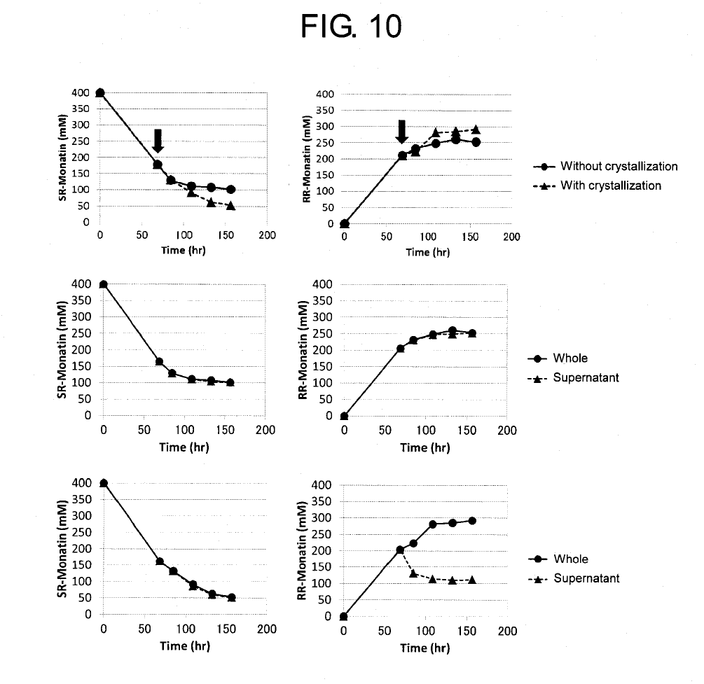

As a result, 179 mM (2S,4R)Monatin and 211 mM (2R, 4R)Monatin were formed after the reaction for 69 hours (FIG. 10).

This reaction solution was adjusted to pH 6.5 with a sulfuric acid, and bubbled with an argon gas. 3 mg/mL of the L-amino acid aminotransferase solution, 5 mg/mL of the D-amino acid aminotransferase solution, and 0.0015 mg/mL of the alanine racemase solution (Unitika) were added to 2.8 mL of the reaction solution to make a total volume 3 mL. The L-amino acid aminotransferase solution and the D-amino acid aminotransferase solution were substituted with the Tris-HCl buffer (pH 7.0) prior to the addition. In an experimental run in which preferential crystallization was performed simultaneously, 150 mM magnesium sulfate heptahydrate was dissolved in this reaction solution and 0.004 g of the seed crystal [the crystal of the ((2R,4R)Monatin)₂ magnesium salt having the same crystal form as that of the crystal obtained in Reference Example 5] was added to the reaction solution. The reaction was carried out using a 15 mL Falcon tube at 25° C. for additional 88 hours with stirring. 990 μL of TE buffer was added to a sampled reaction solution (10 μl) and a supernatant (10 μL) obtained by centrifuging the reaction solution, the mixture was ultrafiltrated using Amicon Ultra 0.5 mL 10 k (Millipore), and the resulting filtrates were analyzed. HPLC was used for the analysis. HPLC was carried out under the condition described in Example 5.

As a result, the concentration of the (2R,4R)Monatin in the reaction solution was 253 mM in the case of the enzymatic reaction alone (FIG. 10, middle panel) whereas its concentration was 292 mM in the case where the enzymatic reaction and the preferential crystallization were performed simultaneously (FIG. 10, lower panel). In the case where the enzymatic reaction and the preferential crystallization were performed simultaneously, the concentration of the (2R,4R)Monatin in the supernatant of the reaction solution was 111 mM (FIG. 10, lower panel), and the crystal was precipitated. Therefore, it was demonstrated that the amount of the accumulated (2R,4R)Monatin in the reaction solution was increased by simultaneously performing the reaction of forming the (2R, 4R)Monatin and the preferential crystallization of the ((2R,4R)Monatin)₂ magnesium salt

The resulting slurry solution was centrifuged, the obtained crystal was washed with water in a small amount, and the wet crystal was dried at 40° C. under reduced pressure to yield 0.216 g of (2R,4R)Monatin. The obtained crystal, mother solution, and washing solution were analyzed by HPLC to determine its yield and analyze its quality. As a result, it was confirmed that a crystal of a ((2R,4R)Monatin)₂ magnesium salt had been obtained. HPLC was carried out under the condition described above (HPLC analysis condition).

(2R,4R)Monatin content: 58.7% by weight
(2S,4R)Monatin content: 2.6% by weight
Magnesium content: 2.2% by weight
Potassium content: 1.8% by weight
Water content: 18.0% by weight

### Example 8

### Isomerization Reaction from (2S,4R)Monatin to (2R,4R)Monatin Using Racemase

An isomerization reaction using the racemase Rac39 prepared in the same manner as in Example 2 was carried out on a scale of 50 mL. A reaction solution composed of 100 mM Tris-HCl buffer, 100 mM (2S,4R)Monatin, 50 μM PLP, and 0.016 U/mL Rac39 was prepared, and reacted at 33° C. for 65 hours with shaking at 120 rpm. An aliquot of the sample reacted after 65 hours was collected, and its reaction was stopped by adding a sodium citrate solution (pH 4.5). The reaction solution after stopping the reaction was centrifuged to obtain a supernatant, which was then subjected to the HPLC analysis. As a result, 40.1 mM (2R, 4R)Monatin was accumulated.

### Example 9

### Preferential Crystallization of (2R,4R)Monatin From Enzyme Reaction Solution

0.45 g (2.2 mmol) of magnesium chloride hexahydrate was dissolved in 45 mL of the enzyme reaction solution obtained in Example 8, and concentrated under reduced pressure. 7.7 g of the resulting concentrated solution was filtrated with a 0.2 μm filter, 2.4 mg of the seed crystal [the crystal of the ((2R,4R)Monatin)₂ magnesium salt having the same crystal form as that of the crystal obtained in Reference Example 5] was added to the resulting filtrate, and the mixture was stirred at 25° C. for 24 hours. The resulting slurry solution was filtrated, a yielded crystal was washed with 0.5 g of water, and the wet crystal was dried at 40° C. under reduced pressure to yield 0.41 g of (2R,4R)Monatin. The obtained crystal, mother solution, and washing solution were analyzed by HPLC to analyze determine its yield and analyze its quality. As a result, it was confirmed that a crystal of ((2R,4R)Monatin)₂ magnesium salt had been obtained.

HPLC area purity (210 nm): 91.5%
Water content: 16.3% by weight
Magnesium content: 3.72% by weight

### Example 10

### Facilitation of Preferential Crystallization by Addition of Organic Solvent to Reaction Solution

A 300 mL four-necked flask was purged with argon, then 58.0 g of water, 20.0 g (68.4 mmol) of free form of (2S, 4R) monatin, 1.58 g (30.7 mmol) of magnesium hydroxide, and 0.850 g (6.96 mmol) of salicylaldehyde were added thereto, and the mixture was reacted at 65° C. for 24 hours with heating and stirring. Subsequently, 19 mg of the seed crystal [the crystal of the ((2R,4R)Monatin)₂ magnesium salt having the same crystal form as that of the crystal obtained in Reference Example 5] was added, then 19.3 g of methanol was added over one hour, and the mixture was heated and stirred for 48 hours. Further, 38.7 g of methanol was added over one hour, and the mixture was heated and stirred for 24 hours. The resulting slurry solution was cooled to 25° C., filtrated, and a crystal was washed with 10.0 g of 50% methanol. The obtained wet crystal was dried at 40° C. under reduced pressure to yield 19.0 g (54.5 mmol) of a crystal of a ((2R,4R)Monatin)₂ magnesium salt. From characteristic X ray diffraction peaks, it was confirmed that the obtained crystal of the ((2R,4R)Monatin)₂ magnesium salt had the same crystal form as that of the crystal obtained in Reference Example 4.

(2R,4R)Monatin yield: 79.7%
(2R,4R)Monatin content: 83.7% by weight
Characteristic X ray diffraction peaks (2θ±0.2°, CuKα): 4.9°, 16.8°, 18.0°, and 24.6°

### Reference Example 6

### Preparation of Crystal of Free Form of (2R,4R)Monatin from Crystal of ((2R,4R)Monatin)₂ Magnesium Salt

15 g (46.3 mmol) of the crystal of the ((2R,4R)Monatin)₂ magnesium salt obtained in Example 10 was dispersed in 285 mL of water, and 24.2 g of 1 M sulfate was added and the mixture was stirred for 18 hours with keeping temperature at 10° C. A formed slurry was separated, and 29.3 g of the resulting wet crystal was dried under reduced pressure at 40° C. to yield 13.1 g of a crystal of a free form of (2R,4R)Monatin.

### Purity: 99%

Magnesium: 100 ppm or less.

### Reference Example 7

### Preparation of Crystal of (2R,4R)Monatin Potassium Salt from Crystal of Free Form of (2R, 4R)Monatin

10 g (33.9 mmol) of the crystal of the free form of (2R,4R)Monatin obtained in Reference Example 6 was dispersed in 10 g of water, and 3.9 g (33.9 mmol) of an aqueous solution of 50% potassium hydroxide was added thereto and dissolved therein. 58 g of methanol was added over 3 hours with keeping the temperature at 40° C., and then the mixture was stirred at 25° C. for 1.5 hours. A formed slurry was separated. 13.1 g of the resulting wet crystal was dried under reduced pressure at 40° C. to yield 10.2 g of a crystal of a (2R,4R)Monatin potassium salt.

### Purity: 99%

Water content: 6.1% by weight
Potassium content: 12.5% by weight

### Example 11

### Facilitation of Preferential Crystallization by Adding Organic Solvent to Reaction Solution

A 1000 mL four-necked flask was purged with argon gas, and 290.7 g of water, 100.0 g (342.1 mmol) of a free (2S, 4R)Monatin, 9.26 g (154.0 mmol) of magnesium hydroxide, 3.47 g (17.1 mmol) of magnesium chloride hexahydrate, and 4.26 g (34.5 mmol) of salicylaldehyde were added thereto. The mixture was stirred with heating at 65° C. for 24 hours. Subsequently, 5.50 g (17.1 mmol) of a seed crystal [the crystal of ((2R,4R)Monatin)₂ magnesium salt having the same crystal form as that of the crystal obtained in Reference Example 3] was added, subsequently 33.5 g of 2-propanol was added over 6 hours, and then the mixture was stirred with heating for 9 hours. Subsequently, 63.4 g of 2-propanol was added over 3 hours and then the mixture was stirred with heating for 30 hours. Further, 193.8 g of 2-propanol was added over 2 hours, and then the mixture was continuously stirred with heating for 22 hours. A resulting slurry solution was cooled to 25° C. over 4 hours, filtered and the crystal was washed with 50.0 g of 50% 2-propanol. The resulting wet crystal was dried under reduced pressure at 40° C. to yield 102.9 g (292.4 mmol) of a crystal of a ((2R,4R)Monatin)₂ magnesium salt. From characteristic X ray diffraction peaks, it was confirmed that the obtained crystal of the ((2R,4R)Monatin)₂ magnesium salt had the same crystal form as that of the crystal obtained in Reference Example 4.

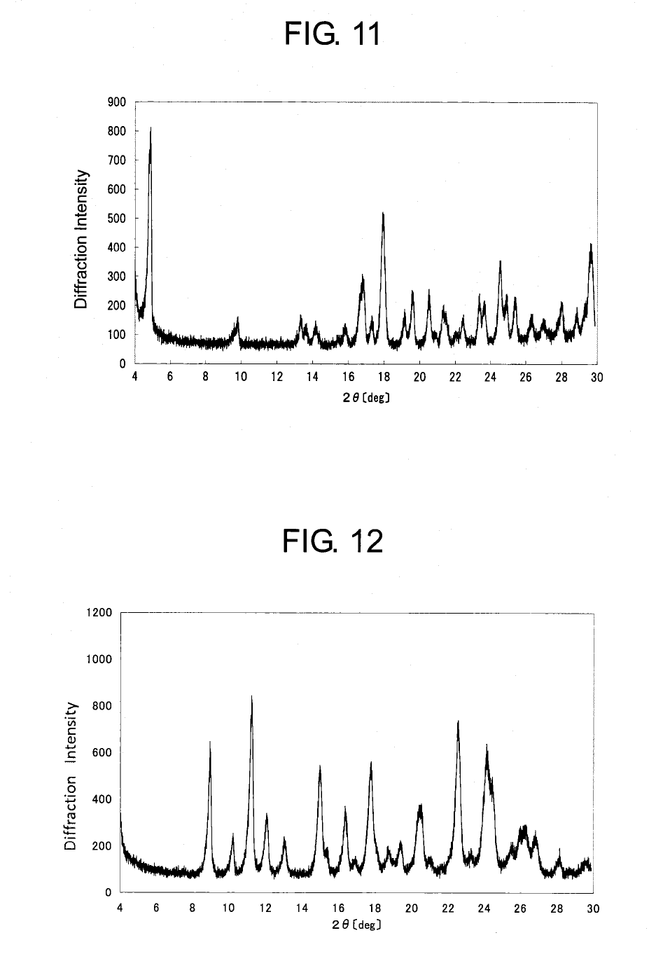

(2R,4R)Monatin yield: 81.4%
(2R,4R)Monatin content: 83.0% by weight
Characteristic X ray diffraction peaks (2θ±0.2°, CuKα): 4.9°, 16.8°, 18.0° and 24.6° (FIG. 11).

### INDUSTRIAL APPLICABILITY

It has become possible to provide a method of efficiently producing a (2R,4R)Monatin multivalent metal salt that has a good sweetness property and is excellent in storage stability by allowing an aldehyde or a racemase to be acted on a solution containing (2S,4R)Monatin in the presence of a multivalent metal ion. This makes it possible to significantly provide various food products, various pharmaceutical products, and various products containing the (2R,4R)Monatin multivalent metal salt.

1. A method of producing a crystal of a (2R,4R)Monatin multivalent metal salt, comprising contacting (2S,4R)Monatin with an aldehyde or one or two or more enzymes capable of forming (2R,4R)Monatin from the (2S,4R)Monatin in an aqueous solution containing a multivalent metal ion to precipitate the crystal of the (2R,4R)Monatin multivalent metal salt.

2. A method of producing a crystal of a (2R,4R)Monatin multivalent metal salt, comprising allowing an aldehyde or one or two or more selected from the group consisting of racemases and aminotransferases to be acted on an aqueous solution containing (2S,4R)Monatin in the presence of a multivalent metal ion to obtain the crystal of the (2R,4R)Monatin multivalent metal salt.

3. The method of producing the crystal of the (2R,4R)Monatin multivalent metal salt according to claim 1, wherein the aldehyde is an aromatic aldehyde.

4. The method of producing the crystal of the (2R,4R)Monatin multivalent metal salt according to claim 1, wherein the enzyme capable of forming the (2R,4R)Monatin from the (2S,4R)Monatin is a racemase.

5. The method of producing the crystal of the (2R,4R)Monatin multivalent metal salt according to claim 4, wherein the racemase comprises the amino acid sequence of SEQ ID NO:2.

6. The method of producing the crystal of the (2R,4R)Monatin multivalent metal salt according to claim 1, wherein the enzyme capable of forming the (2R,4R)Monatin from the (2S,4R)Monatin is an aminotransferase.

7. The method of producing the crystal of the (2R,4R)Monatin multivalent metal salt according to claim 1, wherein the enzyme capable of forming the (2R,4R)Monatin from the (2S,4R)Monatin is an L-amino acid aminotransferase and a D-amino acid aminotransferase.

8. The method of producing the crystal of the (2R,4R)Monatin multivalent metal salt according to claim 1, wherein the enzyme capable of forming the (2R,4R)Monatin from the (2S,4R)Monatin is an L-amino acid aminotransferase, a D-amino acid aminotransferase, and a racemase.

9. The method of producing the crystal of the (2R,4R)Monatin multivalent metal salt according to claim 7, wherein the L-amino acid aminotransferase comprises the amino acid sequence of SEQ ID NO:4 and the D-amino acid aminotransferase comprises the amino acid sequence of SEQ ID NO:6.

10. The method of producing the crystal of the (2R,4R)Monatin multivalent metal salt according to claim 1, wherein the multivalent metal is a bivalent alkaline earth metal.

11. The method of producing the crystal of the (2R,4R)Monatin multivalent metal salt according to claim 10, wherein the alkaline earth metal is magnesium.

12. The method of producing the crystal of the (2R,4R)Monatin multivalent metal salt according to claim 1, wherein a pH value of the aqueous solution is 4 to 11.

13. The method of producing the crystal of the (2R,4R)Monatin multivalent metal salt according to claim 1, wherein an organic solvent in an amount of 5% by volume or less is present in the aqueous solution.

14. The method of producing the crystal of the (2R,4R)Monatin multivalent metal salt according to claim 1, wherein the crystal has characteristic X ray diffraction peaks at 8.9°, 11.2°, 15.0°, 17.8°, and 22.5° as diffraction angles (2θ±0.2°, CuKα).

15. The method of producing the crystal of the (2R,4R)Monatin multivalent metal salt according to claim 1, wherein the crystal has characteristic X ray diffraction peaks at 4.9°, 16.8°, 18.0°, and 24.6° as diffraction angles (2θ±0.2°, CuKα).

16. The method of producing the crystal of the (2R,4R)Monatin multivalent metal salt according to claim 1, comprising collecting the crystal of the (2R,4R)Monatin multivalent metal salt.

17. The method of producing the crystal of the (2R,4R)Monatin multivalent metal salt according to claim 1, wherein an amount of the formed (2R,4R)Monatin is allowed to be increased by simultaneously performing an isomerization from the (2S,4R)Monatin to the (2R,4R)Monatin and a crystallization of the (2R,4R)Monatin multivalent metal salt.

18. The method of producing the crystal of the (2R,4R)Monatin multivalent metal salt according to claim 1, further comprising facilitating the precipitation of the crystal of the (2R,4R)Monatin multivalent metal salt by adding an organic solvent to the aqueous solution after forming the (2R,4R)Monatin.
